(19) United States
(12) Patent Application Publication
(10) Pub. No.: US 2019/0022262 A1
(43) Pub. Date: Jan. 24, 2019

(54) NEUTRALIZING SYSTEM

(71) Applicant: ScentLok Technologies, Inc., Muskegon, MI (US)

(72) Inventors: Nicholas C. Andrews, Muskegon, MI (US); Greg Paquin, Muskegon, MI (US)

(21) Appl. No.: 16/042,873

(22) Filed: Jul. 23, 2018

Related U.S. Application Data
(60) Provisional application No. 62/536,354, filed on Jul. 24, 2017.
(60) Provisional application No. 62/657,516, filed on Apr. 13, 2018.

(51) Int. Cl. A61L 2/20; A45C 15/00; B01D 53/02; A61L 9/16

(52) U.S. Cl. CPC A61L 2/202; A45C 15/00; B01D 53/02; A61L 9/16; A61L 2202/15; B01D 2253/108; B01D 2257/90; A61L 2202/26; A61L 2202/11; B01D 2253/102

(57) ABSTRACT

A bag includes at least one panel, an emitter configured to provide a supply of neutralizer, and a gas distribution assembly coupled to the at least one panel. The at least one panel defines an enclosed volume configured to receive at least one item and an opening configured to at least selectively facilitate access to the enclosed volume. The gas distribution assembly defines a gas distribution volume extending along the at least one panel and a series of apertures. The apertures are positioned along a length of the gas distribution volume and configured to fluidly couple the gas distribution volume to the enclosed volume. The gas distribution volume is fluidly coupled to the emitter such that the neutralizer is configured to flow from the emitter, through the gas distribution volume, and into the enclosed volume.

### CROSS-REFERENCE TO RELATED PATENT APPLICATIONS

This application claims the benefit of U.S. Provisional Application No. 62/536,354, filed Jul. 24, 2017, and U.S. Provisional Application No. 62/657,516, filed Apr. 13, 2018, both of which are incorporated herein by reference in their entireties.

### BACKGROUND

Humans produce, expel, facilitate the growth of, or otherwise provide substances that emit a spectrum of odors in the form of odor particles. The rate of production of such substances, and by extension the magnitude of the odors that they emit, increases in hot conditions or when humans participate in exercise. At low magnitudes, the odors produced by such substances can be detected by the acute senses of smell of various animals. Hunters, field biologists, nature photographers, and others rely on remaining undetected by target species (e.g., deer, bear) while hunting in a natural environment, or in other applications. At greater magnitudes, the odors can be detected by humans and are often considered unpleasant.

Upon coming into contact with humans or animals, fabrics and other absorbent materials (e.g., used in clothing, athletic equipment, furniture, etc.) contact and retain odor-emitting substances. Further, fabrics and other absorbent materials may contact and retain odor-emitting substances, bacteria, dust mites, or other undesirable organisms or substances even without directly contacting humans. Such undesirable organisms and substances often remain active for large period of time or until the affected item is washed. Some items (e.g., beds, protective padding, clothing, etc.) are difficult to wash or may be damaged by washing, and accordingly are not washed regularly.

### SUMMARY

One embodiment of the present disclosure relates to a bag including at least one panel, an emitter configured to provide a supply of neutralizer, and a gas distribution assembly coupled to the at least one panel. The at least one panel defines an enclosed volume configured to receive at least one item and an opening configured to at least selectively facilitate access to the enclosed volume. The gas distribution assembly defines a gas distribution volume extending along the at least one panel and a series of apertures. The apertures are positioned along a length of the gas distribution volume and configured to fluidly couple the gas distribution volume to the enclosed volume. The gas distribution volume is fluidly coupled to the emitter such that the neutralizer is configured to flow from the emitter, through the gas distribution volume, and into the enclosed volume.

Another embodiment relates to an assembly for neutralizing substances or organisms. The assembly includes an enclosure defining an enclosed volume, an emitter configured to provide a supply of neutralizer, and a gas distribution assembly including a conduit extending within the enclosure and coupled to the enclosure. The enclosure has a top side, a bottom side, and a third side extending between the top side and the bottom side. The conduit defines a gas distribution volume extending along at least one of the top side and the third side of the enclosure. The gas distribution assembly further defines a series of apertures positioned along a length of the conduit that fluidly couple the gas distribution volume to the enclosed volume. The gas distribution volume is fluidly coupled to the emitter such that the neutralizer is configured to flow from the emitter, through the gas distribution volume, and into the enclosed volume.

Yet another embodiment relates to a neutralizing system including an emitter including an ozone generator configured to provide a supply of ozone and a gas distribution assembly including a conduit. The conduit defines a gas distribution volume extending along a length of the conduit. The gas distribution assembly defines a series of outlets fluidly coupled to the gas distribution volume and positioned along the length of the conduit. The gas distribution volume is fluidly coupled to the emitter such that the gas distribution assembly is configured to expel the ozone from the gas distribution volume at multiple locations along the length of the conduit.

Alternative exemplary embodiments relate to other features and combinations of features as may be generally recited in the claims.

### BRIEF DESCRIPTION OF THE FIGURES

The disclosure will become more fully understood from the following detailed description, taken in conjunction with the accompanying figures, in which:

FIG. 1 is a front perspective view of a bag, according to an exemplary embodiment;

FIG. 2 is a rear perspective view of the bag of FIG. 1;

FIG. 3 is left side perspective view of the bag of FIG. 1;

FIG. 4 is a perspective view of a first pocket of the bag of FIG. 1, according to an exemplary embodiment;

FIG. 5 is a top perspective view of the bag of FIG. 1 in an open configuration;

FIG. 6 is a front perspective view from inside of the bag of FIG. 1;

FIG. 7 is a rear perspective view from inside of the bag of FIG. 1;

FIG. 8 is a schematic view of a neutralizing system of the bag of FIG. 1, according to an exemplary embodiment;

FIG. 9 is a partial section view of a gas distribution assembly of the neutralizing system of FIG. 8, according to an exemplary embodiment;

FIG. 10 is a left side perspective view from inside of the bag of FIG. 1;

FIG. 11 is a right side perspective view from inside of the bag of FIG. 1;

FIG. 12 is a section view of the gas distribution assembly of FIG. 9;

FIG. 13 is a perspective view of a second pocket of the bag of FIG. 1, according to an exemplary embodiment;

FIG. 14 is a perspective view of a pet bed including the neutralizing system of FIG. 8, according to an exemplary embodiment;

FIG. 15 is a perspective view of a mattress including the neutralizing system of FIG. 8, according to an exemplary embodiment;

FIG. 16 is a perspective view of a pad including the neutralizing system of FIG. 8, according to an exemplary embodiment;

FIG. 17 is a perspective view of a closet including the neutralizing system of FIG. 8, according to an exemplary embodiment;

FIG. 18 is a perspective view of a bag including the neutralizing system of FIG. 8, according to an exemplary embodiment;

FIG. 19 is a perspective view of a tent including the neutralizing system of FIG. 8, according to an exemplary embodiment;

FIG. 20 is a perspective view of a tent including the neutralizing system of FIG. 8, according to another exemplary embodiment;

FIG. 21 is a perspective view of a gas distribution system of the tent of FIG. 20, according to an exemplary embodiment;

FIG. 22 is a perspective view of a seat outfitted with a seat cover including the neutralizing system of FIG. 8, according to an exemplary embodiment;

FIG. 23 is a perspective view of a seat outfitted with a seat cover including the neutralizing system of FIG. 8, according to another exemplary embodiment;

FIGS. 24 and 25 are perspective views of a harness including the neutralizing system of FIG. 8, according to an exemplary embodiment;

FIGS. 26 and 27 are perspective views of a vest including the neutralizing system of FIG. 8, according to an exemplary embodiment;

FIG. 28 is a schematic perspective view of a bag including a neutralizing system, according to another exemplary embodiment;

FIG. 29 is a schematic perspective view of a bag including a neutralizing system, according to another exemplary embodiment; and

FIG. 30 is a detail view of the neutralizing system of FIGS. 28 and 29, according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

According to an exemplary embodiment, a bag includes a number of panels and pockets sewn together to enclose a main volume and a pair of side pocket volumes. The bag includes a number of zippers that selectively prevent items from passing into or out of the main volume and side pocket volumes. The bag further includes a neutralizing system including an emitter, a gas distribution assembly, and a filter. The emitter, which is located in a pocket on an interior surface of the bag, provides a continuous pressurized flow of neutralizer (e.g., ozone) that neutralizes odors and/or neutralizes undesirable substances or organisms. The gas distribution assembly includes a flexible duct that extends along an interior surface of the bag. The duct defines a gas distribution volume that is fluidly coupled to an outlet of the emitter to receive neutralizer from the emitter. The duct further defines a series of apertures along the length of the duct that fluidly couple the gas distribution volume to the main volume, distributing the neutralizer throughout the bag. The duct is sewn into a seam between two panels of the bag. The filter is a piece of material (e.g., carbon) configured to passively adsorb odor particles. The bag further includes a pocket defined proximate a bottom surface of the bag and configured to be accessed from the exterior of the bag. The pocket includes a grommet defining an aperture connecting the inside of the pocket to the main volume. An electrical cord connected to the emitter passes through this aperture and is stored in the pocket. One or more additional grommets define apertures that fluidly couple the side pocket volumes to the main volume.

The neutralizing system may be integrated into other types of devices. The neutralizing system may be integrated into an enclosure, such as a hunting closet, a bag, or a tent. The neutralizing system may provide neutralizer into an enclosed volume defined by the enclosure or may distribute neutralizer outside the enclosure. Alternatively, the neutralizing system may be integrated into a wearable device, such as a vest or harness, or into a seat cover. The neutralizing system may distribute neutralizer around the device and/or inside of the device.

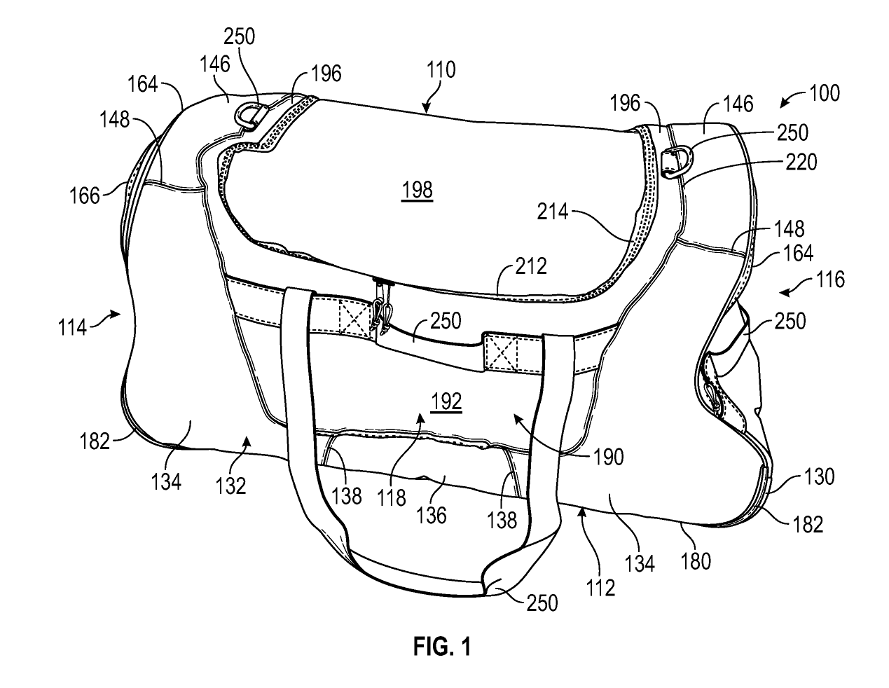

Referring to FIGS. 1-7, a container (e.g., a bag, a duffel bag, a sack, a suitcase, a backpack, a basket, etc.) or enclosure is shown as a bag 100. Although the bag 100 is shown as a duffel bag, it should be understood that the bag 100 may be any type of bag or container. The bag 100 is configured to selectively enclose one or more individual volumes that may be separated (e.g., partially, completely, etc.) from one another (e.g., to facilitate organization). The bag 100 is configured such that the one or more volumes enclosed by the bag 100 can selectively contain one or more items (e.g., clothing, shoes, tools, sports equipment, safety equipment, etc.) to facilitate transport and storage of the items. The quantity and sizes one or more volumes may vary depending upon the intended application of the bag 100.

The bag 100 is formed in part from a flexible material (e.g., fabric, rubberized plastic, etc.) such that the bag 100 can be collapsed (e.g., folded) for storage and expanded as one or more items are added to the one or more volumes enclosed by the bag 100. The bag 100, when fully expanded, is shaped approximately as a rectangular prism. The bag 100 defines a number of sides or surfaces including a top side 110, a bottom side 112, a left side 114, a right side 116, a front side 118, and a rear side 120. The left side 114, the right side 116, the front side 118, and the rear side 120 extend between the top side 110 and the bottom side 112. In other embodiments, the bag 100 is otherwise shaped. By way of example, the bag 100 may be approximately cylindrically shaped.

The bag 100 includes a number of sheets or panels of material that are coupled (e.g., sewn) to one another along the length of one or more exterior edges, forming a number of seams. Each panel may be formed from a different type of material (e.g., depending on where the panel is located in the bag 100). The exact number, shape, and arrangement of the panels and seams may vary throughout different embodiments of the bag 100.

Referring to FIGS. 1-3, the bag 100 includes a panel or sheet, shown as bottom panel 130. The bottom panel 130 extends across the bottom side 112 and bends upward to extend partially up the left side 114 and the right side 116. In some embodiments, the bottom panel 130 is made from a flexible, rubberized material, which prevents the bag from sliding across a surface that supports the bag 100 (e.g., a truck bed). In some embodiments, the bottom panel 130 is non-permeable, preventing the bag 100 from absorbing liquid when set on a wet surface (e.g., wet grass).

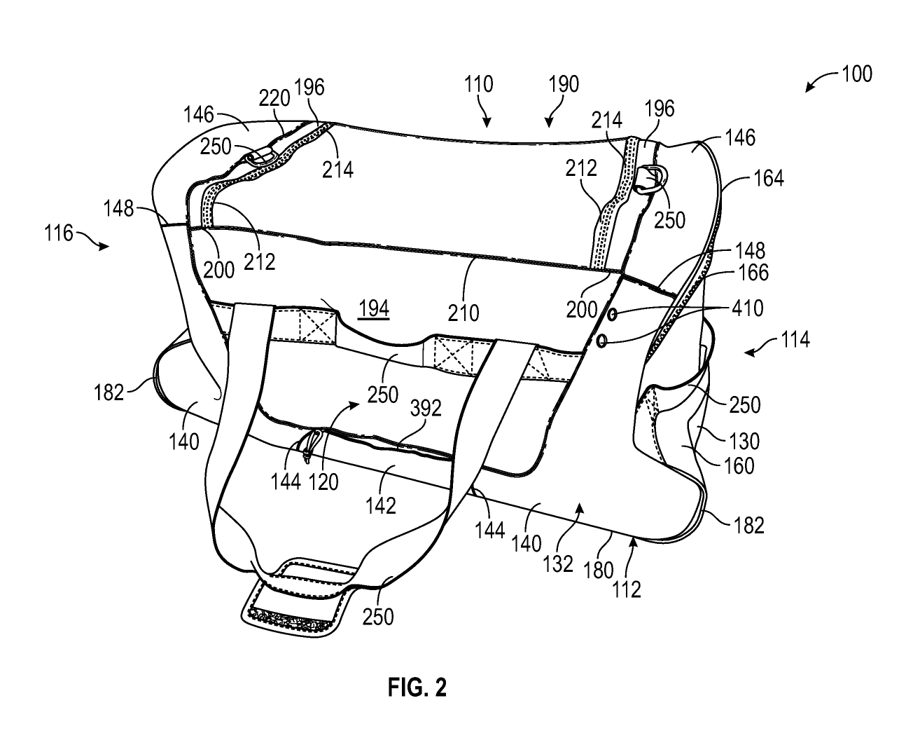

Referring to FIGS. 1 and 2, the bag 100 further includes a panel or panel assembly, shown as outer panel assembly 132. The outer panel assembly 132 extends along the front side 118, the top side 110, and the back side 120. The outer panel assembly 132 includes a pair of side panels 134 and a central panel 136, all extending along the front side 118. The side panels 134 are each coupled to an opposite lateral side of the central panel 136 by a seam 138. The seams 138 are sewn, adhered, or otherwise attached such that the central panel 136 is fixedly coupled to both of the side panels 134. The outer panel assembly 132 further includes a pair of side panels 140 and a central panel 142, all extending along the rear side 120. The side panels 140 are each coupled to an opposite lateral side of the central panel 142 by a seam 144. The seams 144 are sewn, adhered, or otherwise attached such that the central panel 142 is fixedly coupled to both of the side panels 140. The outer panel assembly 132 further includes a pair of top panels 146. The top panels 146 each extend across the top side 110 between one of the side panels 134 and the corresponding side panel 140. A seam 148 couples each side panel 134 to the corresponding top panel 146, and another seam 148 couples each side panel 140 to the corresponding top panel 146. The seams 148 are sewn, adhered, or otherwise attached such that the top panels 146 are fixedly coupled to the corresponding side panel 134 and the corresponding side panel 140. Together, the side panels 134, the central panel 136, the side panels 140, the central panel 142, and the top panels 146 form a continuous structure extending from one edge of the bottom panel 130 to an opposite edge of the bottom panel 130. In some embodiments, one or more components of the outer panel assembly 132 are integrally formed from a single piece of material or broken up into multiple pieces of material.

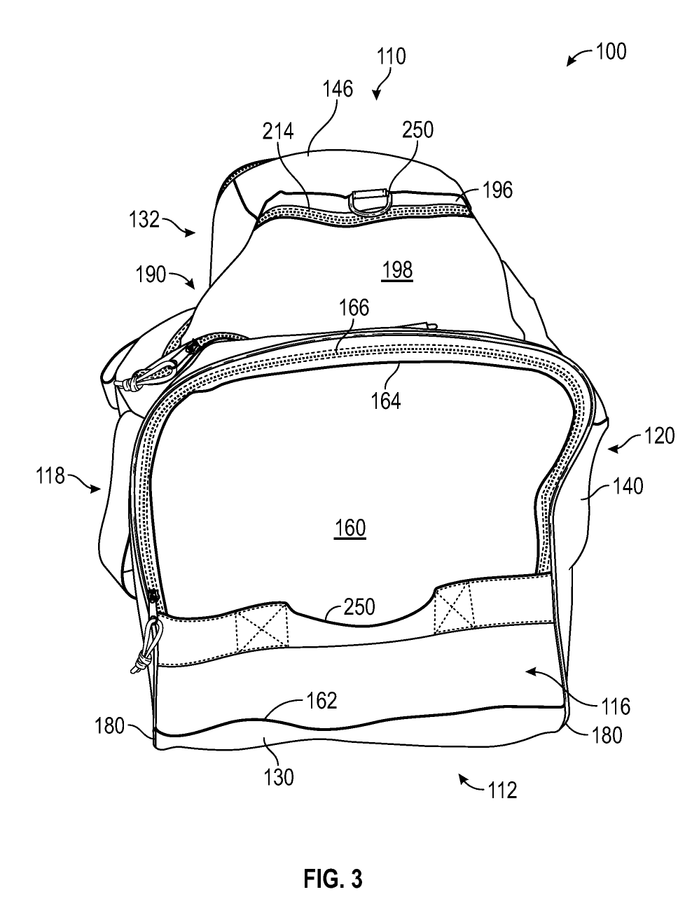

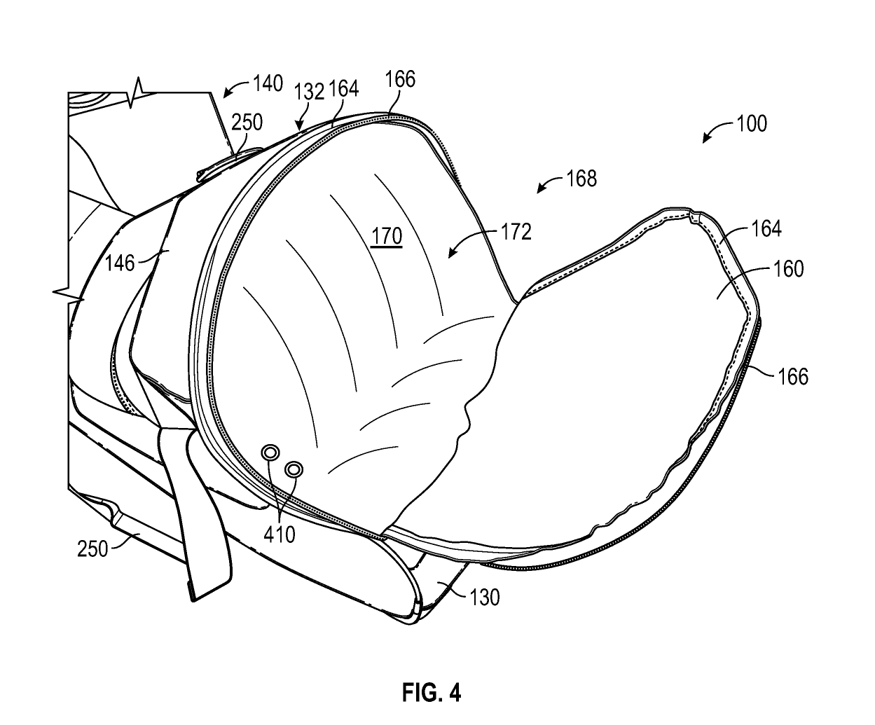

Referring to FIGS. 2-4, the bag 100 further includes a pair of panels or panel assemblies, shown as side panels 160, extending across the left side 114 and the right side 116 of the bag 100. Each of the side panels 160 extends entirely across an opening on either the left side 114 or the right side 116 defined between the bottom panel 130, the side panel 134, the side panel 140, and the top panel 146. The side panels 160 are each coupled to the bottom panel 130 by a seam 162 extending across a bottom edge of each side panel 160. The seams 162 are sewn, adhered, or otherwise attached such that the side panels 160 are fixedly coupled to the bottom panel 130. The side panels 160 are each further coupled to the outer panel assembly 132 by a seam 164 extending along a front, a top, and a rear edge of each side panel 160. Portions of the seam 164 near the bottom of the side panel 160 on both the front edge and the rear edge of each side panel 160 are sewn, adhered, or otherwise attached such that a portion of each side panel 160 is fixedly coupled to the outer panel assembly 132. The remainder of the seam 164 includes a zipper 166. The zipper 166 selectively couples a portion each side panel 160 to the outer panel assembly 132. With one of the zippers 166 in an open position, the corresponding side panel 160 can be separated from the outer panel assembly 132, as shown in FIG. 4, to define an opening 168 between the side panel 160 and the outer panel assembly 132. When in a closed position, shown in FIG. 3, the zipper 166 closes the opening 168.

Referring to FIG. 4, the bag 100 further includes a bag, sack, or pocket, shown as pocket 170, coupled to each side panel 160. Each pocket 170 is sewn into the seam 164 on each side of the zipper 166 such that an edge of the pocket 170 is fixedly coupled to the side panel 160 and the outer panel assembly 132. Each pocket 170 defines a secondary volume, shown as side pocket volume 172. With the zipper 166 in an open position, the side pocket volume 172 can be accessed through the opening 168. With the zipper 166 in a closed position, the side pocket volume 172 is enclosed by the zipper 166, the side panel 160, and the pocket 170, preventing items from entering or exiting the side pocket volume 172. The pockets 170 facilitate storage of items separate from one another (e.g., for organizational purposes, to prevent wet clothes from contacting electronics, etc.). Although only the side panel 160 and pocket 170 of the right side of the bag 100 are shown in FIGS. 3 and 4, it should be understood that the bag 100 includes a similar, mirrored side panel 160 and pocket 170 on the left side.

Referring to FIGS. 1 and 2, a pair of seams 180 extend between the bottom panel 130 and the outer panel assembly 132 on the front side 118 and the rear side 120. The seams 180 are sewn, adhered, or otherwise attached such that the bottom panel 130 is fixedly coupled to the outer panel assembly 132. The portions of the seam 180 nearest the corners of the bottom side 112 (e.g., where the bottom panel 130 bends upward) are covered by guards, shown as edge guards 182. The edge guards 182 are made of a material harder than that of the seam 180 (e.g., vinyl). The edge guards 182 wrap around the seam 180 to protect the seam 180 from damage (e.g., caused by rubbing against an abrasive surface). The edge guards 182 are sewn into the seam 180 such that the edge guards 182 are fixedly coupled to the bottom panel 130 and the outer panel assembly 132. As shown in FIGS. 1-3, the seams 164 and the seams 180 transition into one another (e.g., are aligned with one another, contact one another, are coupled to one another, etc.), forming a continuous loop around the exterior of the bag 100. In some such embodiments, the seams 164 and the seams 180 are each portions of one continuous seam. In other embodiments, the seams 164 and the seam 180 may be separated from one another or broken up into a series of shorter seams.

Referring to FIGS. 1 and 2, the bag 100 further includes a panel or panel assembly, shown as inner panel assembly 190. The inner panel assembly 190 extends across an opening defined by the side panels 134, the central panel 136, the side panels 140, the central panel 142, and the top panels 146 of the outer panel assembly 132. The inner panel assembly 190 includes a front panel 192, a rear panel 194, a pair of top panels 196, and a top flap 198. The front panel 192 extends along the front side 118 between the side panels 134 and the central panel 136. The rear panel 194 extends along the rear side 120 between the side panels 140 and the central panel 142. The top panels 196 extend along the top side 110 between the front panel 192 and the rear panel 194, each extending along an edge of one of the top panels 146. A seam 200 couples the rear panel 194 to each top panel 196. The seams 200 are sewn, adhered, or otherwise attached such that the top panels 196 are fixedly coupled to the front panel 192 and the rear panel 194. The front panel 192 and the top panels 196 are integrally formed from a single piece of material. In other embodiments, the front panel 192 and the top panels 196 are formed from separate pieces of material and fixedly coupled together by additional seams 200.

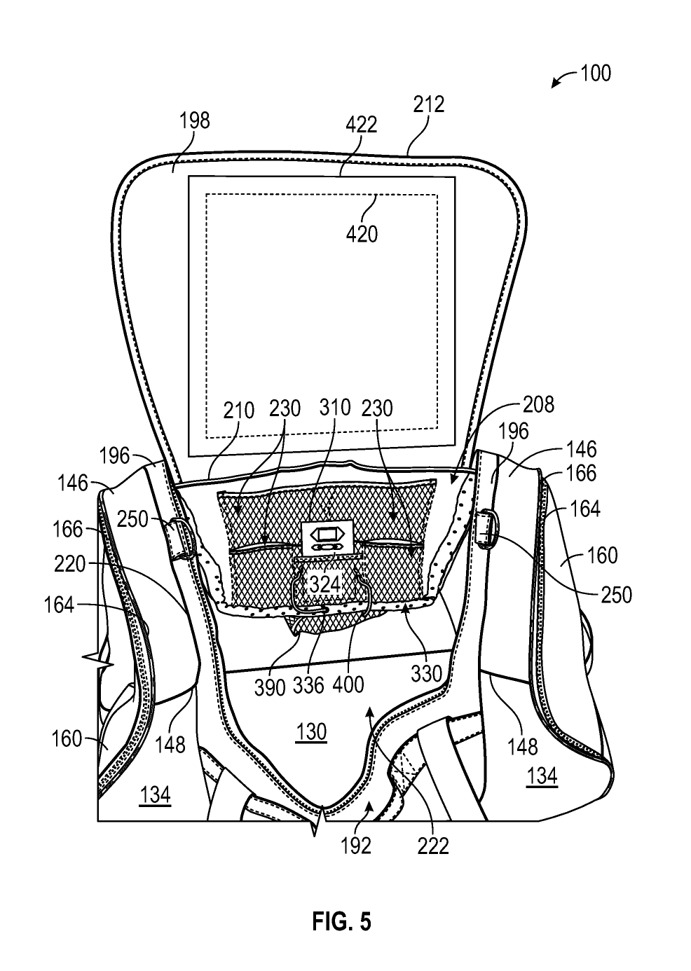

Referring to FIGS. 1, 2, and 5, the top flap 198 extends across an opening 208 defined between the front panel 192, the rear panel 194, and the top panels 196. A seam 210 couples a rear edge of the top flap 198 to a top edge of the rear panel 194. The seam 210 is sewn, adhered, or otherwise attached such that the rear edge of the top flap 198 is fixedly coupled to the rear panel 194. A seam 212 extends along the edges of the top flap 198 adjacent the front panel 192 and the top panels 196. The seam 212 includes a zipper 214 extending along approximately the entire length of the seam 212. The zipper 214 selectively couples the top flap 198 to the front panel 192 and the top panels 196. With the zipper 214 in a closed position, as shown in FIGS. 1 and 2, the top flap 198 extends across the opening 208. With the zipper 214 in an open position, as shown in FIG. 5, the top flap 198 can be separated from the front panel 192 and the top panels 196 to uncover the opening 208. In some embodiments, one or more components of the inner panel assembly 190 are integrally formed from a single piece of material or broken up into multiple pieces of material.

Referring to FIGS. 1 and 2, a seam 220 extends between an inner edge of the outer panel assembly 132 and an outer edge of the inner panel assembly 190. A portion of the seam 220 is sewn, adhered, or otherwise attached such that the front panel 192 is fixedly coupled to the side panels 134 and the central panel 136, the rear panel 194 is fixedly coupled to the side panels 140, and the top panels 196 are fixedly coupled to the top panels 146. Together, the bottom panel 130, the outer panel assembly 132, the side panels 160, and the inner panel assembly 190 enclose an enclosed volume. The enclosed volume includes the side pocket volumes 172, shown in FIG. 4, and a main volume 222, shown in FIG. 5. The main volume 222 is selectively accessible through the opening 208. With the zipper 214 in a closed position, the top flap 198 prevents items from entering or exiting the main volume 222. With the zipper 214 in an open position, the top flap 198 can be moved away from the front panel 192 and the top panels 196 to facilitate access to the main volume 222 through the opening 208. The opening 208 is positioned such that the main volume 222 can be accessed through the opening 208 when the bottom side 112 of the bag 100 rests on a support surface (e.g., the ground) such that the bag 100 is upright.

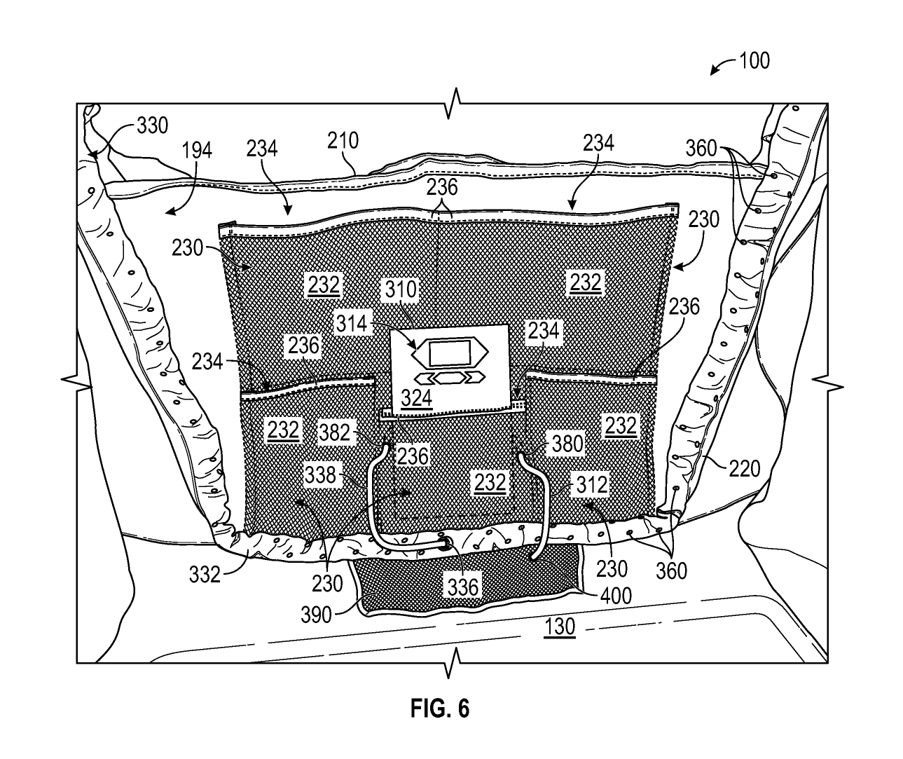

In some embodiments, the bag 100 further includes a series of pockets, pouches, or dividers arranged within the main volume 222. Such pockets facilitate further organization of items within the main volume 222. As shown in FIG. 6, the bag 100 includes a series of pockets 230 coupled to an interior surface of the rear panel 194. The pockets 230 each include a wall 232 formed from a sheet of material (e.g., fabric, mesh, canvas, etc.) The wall 232 is coupled (e.g., sewn) to the rear panel 194 along a bottom edge, a left edge, and a right edge of the pocket 230, forming the pocket 230. In some embodiments, one sheet of material forms the walls 232 of multiple pockets 230 arranged adjacent one another. A portion of the top edge of each pocket 230 is left uncoupled from the rear panel 194 or is selectively coupled to the rear panel 194 to define an opening 234, through which an interior volume of the pocket 230 can be accessed. In some embodiments, one or more of the pockets 230 includes a closure member, shown as elastic band 236, arranged along the top edge of the pocket 230. The elastic band 236 is coupled (e.g., sewn) to the rear panel 194, producing a biasing force that biases a top edge of the pocket 230 toward the rear panel 194. In other embodiments, one or more of the pockets 230 includes other types of closure members, such as zippers or buttons. The elastic band 236 partially or completely closes the opening 234 of each pocket 230 to facilitate holding items within the pockets 230.

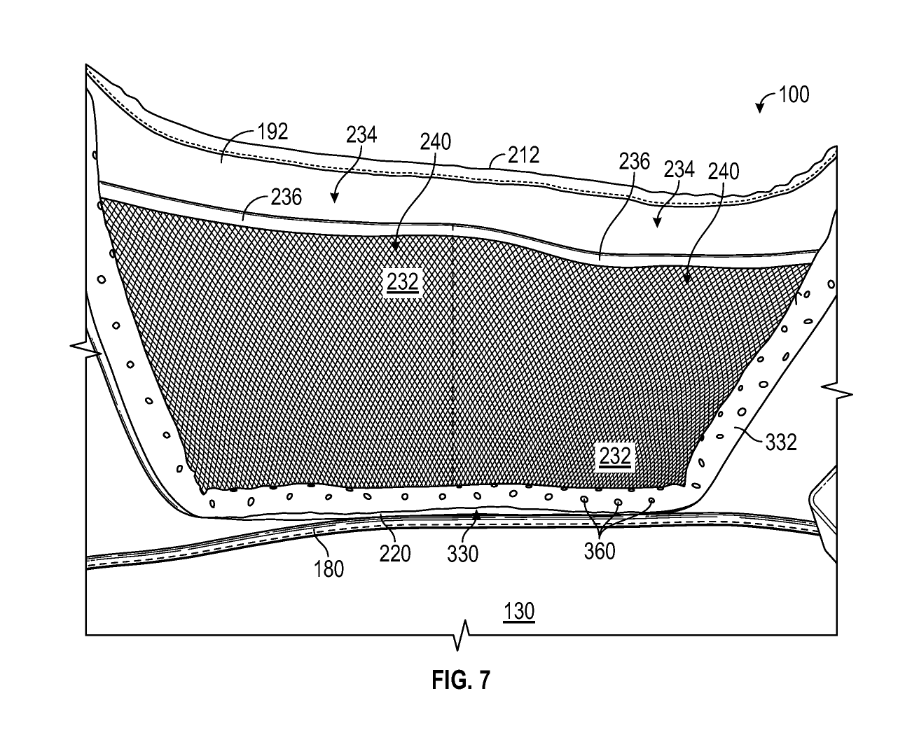

Referring to FIG. 7, the bag 100 further includes a set of pockets 240. The pockets 240 may be substantially similar to the pockets 230 except that the pockets 240 each include a wall 232 that is coupled to the front panel 192 instead of the rear panel 194. The exact shape, arrangement, and quantity of the pockets 230 and the pockets 240 vary throughout different embodiments of the bag 100.

Referring to FIGS. 1 and 2, the bag 100 includes a number of handles, straps, hooks, or rings, shown as interfaces 250. The interfaces 250 facilitate a user manipulating (e.g., pulling, pushing, grabbing, carrying, etc.) the bag 100. Additionally, the interfaces 250 facilitate distributing the weight of the bag 100 on a user carrying the bag 100. By way of example, the interface 250 may be a strap configured to apply the weight of the bag 100 on a shoulder of a user. The interfaces 250 may be coupled (e.g., sewn, adhered, etc.) to any of the panels and/or any of the seams of the bag 100.

Referring to FIG. 6, the bag 100 includes an assembly, shown as neutralizing system 300. The neutralizing system 300 is configured to adsorb, absorb, react with, destroy, disguise, neutralize, and/or otherwise modify odor-emitting substances, odor particles, bacteria, dust mites, and/or other undesirable substances or organisms. The neutralizing system 300 includes a neutralizer emitter, shown as emitter 310, fluidly coupled to a gas distribution assembly 330. The emitter 310 pumps a pressurized neutralizer (e.g., ozone) through the gas distribution assembly 330. The gas distribution assembly 330 is in direct fluid communication with the main volume 222 and evenly distributes the neutralizer throughout the main volume 222 through a series of outlets 360, neutralizing odors, undesirable substances, and/or undesirable organisms throughout the bag 100. In some embodiments, the neutralizing system 300 further includes a filter 420 configured to passively adsorb odor particles present in the bag 100.

The neutralizing system 300 includes compressor, pump, and/or gas generator shown as emitter 310, configured to consume electrical energy and produce, compress, pump, and/or otherwise provide a pressurized supply of neutralizer. The neutralizer may include any type of gas and/or aerosol (e.g., fine particles of liquid dispersed throughout a gas) capable of reacting with, destroying, disguising, or otherwise neutralizing odor particles, odor-emitting substances, bacteria, dust mites, and/or other undesirable substances or organisms. In some embodiments, the emitter 310 is configured to be powered by an electrical outlet (e.g., connected to the electrical grid, powered by a generator, powered by an inverter electrically coupled to a battery, etc.). Accordingly, the emitter 310 includes an electrical cord 312 that is configured to interface with an electrical outlet (e.g., a standard household electrical outlet) and transfer electrical energy to the emitter 310. In other embodiments, the emitter 310 is powered by an energy storage device (e.g., a battery, a fuel cell, etc.) that is stored within the bag 100 and/or the emitter 310. The emitter 310 may further include a user interface 314 configured to facilitate a user issuing commands to the emitter 310 and/or the emitter 310 providing information to the user. As shown in FIG. 6, the user interface 314 includes a screen and a series of buttons. In other embodiments, the user interface 314 includes one or more touch screens, switches, sliders, dials, and other components. The emitter 310 may further include any other components that facilitate generation, pressurization, or storage of the neutralizer or components thereof (e.g., valves, hoses, storage tanks, pressure regulators, fittings, etc.). The pressurized neutralizer passes out of emitter 310 through an outlet 316.

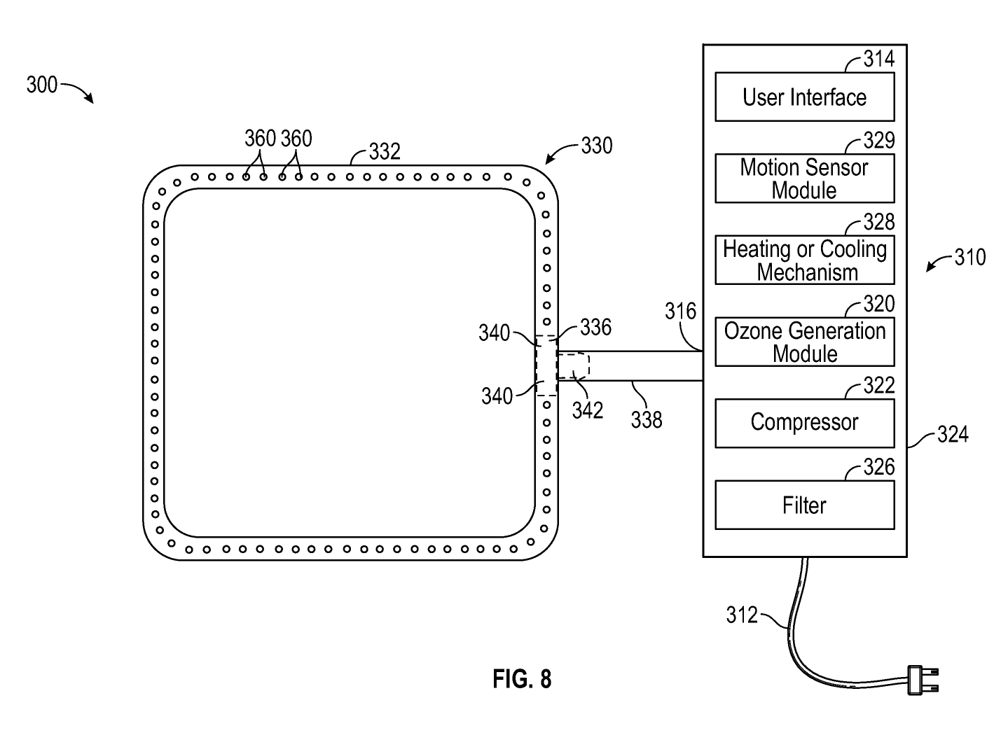

In some embodiments, such as the embodiment shown in FIG. 8, the emitter 310 is an ozone generator configured to generate and pressurize a supply of gaseous ozone (i.e., O₃) that acts as the neutralizer. Ozone is known to neutralize odor particles, odor-causing bacteria, dust mites, and other undesirable substances and organisms. In such embodiments, the emitter 310 includes an ozone generation module 320 configured to convert diatomic oxygen (i.e., O₂) from the surrounding air into ozone. The ozone generation module 320 may be a conventional ozone generator that is configured to utilize a known method of converting diatomic oxygen into ozone (e.g., corona discharge, ultraviolet radiation, electrolysis, etc.). In such embodiments, the emitter 310 further includes a compressor 322 configured to pressurize the generated ozone. The compressor 322 may be any conventional type of compressor. The ozone generation module 320, the compressor 322, and the various other components of the emitter 310 are contained within a housing 324. The housing 324 may further include a plug, outlet, or fitting with which to interface with the electrical cord 312. Alternatively, the electrical cord 312 may be omitted, and an electrical connector may be incorporated directly into the housing 324. The electrical connector may be configured to interface with an electrical outlet directly, or the electrical connector may be configured to interface with an extension cord that, in turn, connects to an electrical outlet. Additionally or alternatively, an energy storage device (e.g., a battery, a capacitor, etc.) may be supplied within the housing 324 to facilitate portability of the emitter 310.

In other embodiments, the emitter 310 is configured to provide another type of neutralizer. By way of a first example, the emitter 310 may include an air ionizer configured to electrically charge (i.e., ionize) air molecules, producing ions that remove undesirable substances from the air. By way of another example, the emitter 310 may provide a pressurized supply of perfume or another type of scented substance that disguises odor molecules present in the air. In such an example, the emitter 310 may include one or more storage containers configured to hold a concentrated supply of the perfume that is later mixed with air to produce the neutralizer. By way of another example, the emitter 310 may be configured to provide a supply of odor oxidizer. In some embodiments, as shown in FIG. 8, the emitter 310 includes a filter 326 made from a filter material. The filter material may include activated carbon, zeolite, nano materials, or another type of filter material. The filter 326 may be configured to adsorb or absorb undesirable substances or organisms from the air that enters the emitter 310 and/or from the neutralizer that exits the emitter 310. The addition of the filter 326 may further facilitate the neutralizing system 300 neutralizing undesirable substances or organisms.

In some embodiments, as shown in FIG. 8, the emitter 310 includes a heating or cooling mechanism 328 configured to provide a pressurized supply of warm or cool air. Such embodiments may be used to heat or cool items contained within the bag 100. By way of example, the bag 100 may be used in cold climates to warm clothing (e.g., hats, gloves, socks, boots, jackets, etc.) that is kept in a cold vehicle (e.g., during a winter hunting expedition). Accordingly, the heating or cooling mechanism 328 may include a heating mechanism (e.g., a resistance heater), a cooling mechanism (e.g., a refrigeration circuit), and/or a heat exchanging mechanism (e.g., a heat sink) to facilitate warming or cooling air.

In some embodiments, as shown in FIG. 8, the emitter 310 includes a motion detector assembly, shown as motion sensor module 329. The motion sensor module 329 is configured to detect the movement of objects (e.g., humans, animals, inanimate objects, etc.) near the emitter 310 (e.g., within a line of sight of the emitter 310, etc.). The motion sensor module 329 may include a passive infrared sensor, a microwave sensor, an ultrasonic sensor, a video camera, an accelerometer, or another type of sensor configured to detect motion.

The motion sensor module 329 can be configured to control operation of the emitter 310 based on the detected motion. By way of example, the motion sensor module 329 may be configured to activate the ozone generation module 320 and/or the compressor 322 in response to the motion sensor module 329 detecting motion. The motion sensor module 329 may additionally or alternatively be configured to deactivate the ozone generation module 320 and/or the compressor 322 in response to the motion sensor module 329 not detecting motion for a threshold period of time. Accordingly, the emitter 310 may be configured to emit neutralizer when a user is present and stop emitting neutralizer when a user is not present, conserving electrical energy. By way of another example, the motion sensor module 329 may be configured to deactivate the ozone generation module 320 and/or the compressor 322 in response to the motion sensor module 329 detecting motion. The motion sensor module 329 may additionally or alternatively be configured to activate the ozone generation module 320 and/or the compressor 322 in response to the motion sensor module 329 not detecting motion for a threshold period of time. Accordingly, the emitter 310 may be configured to stop emitting neutralizer when a user is present and emit neutralizer when a user is not present, reducing or eliminating contact between the neutralizer and the user.

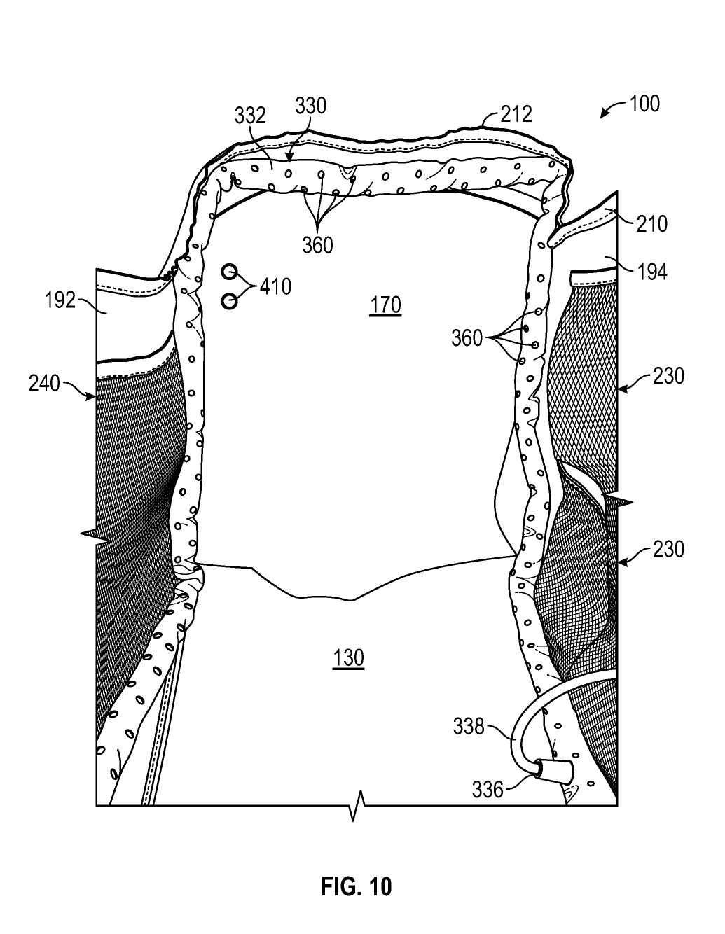

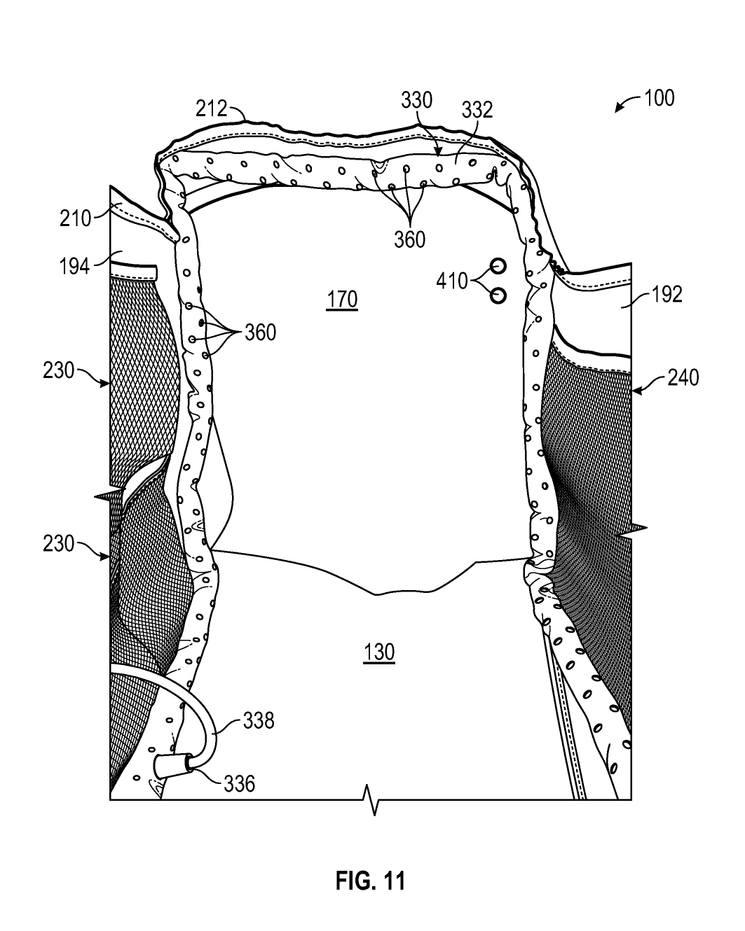

Referring to FIGS. 6-11, the outlet 316 of the emitter 310 is fluidly coupled to a conduit assembly or ducting assembly, shown as gas distribution assembly 330, disposed within the main volume 222 of the bag 100. The gas distribution assembly 330 includes a hose, conduit, duct, or tube, shown as duct 332. The duct 332 is formed from a sheet of material that is folded over and engaging itself (e.g., sealed against itself, sewn together, etc.), enclosing a gas distribution volume 334. The gas distribution volume 334 extends along the length of the duct 332. The duct 332 is made from one or more sheets of woven fabric. The fabric is flexible, facilitating collapsing the bag 100 for storage without resistance from the duct 332.

The gas distribution volume 334 is fluidly coupled to the outlet 316 through an inlet fitting 336 and a hose 338. The inlet fitting 336 extends through the duct 332 and into the gas distribution volume 334. As shown in FIG. 8, the inlet fitting 336 is a T-shaped fitting having two legs 340 extending parallel to the duct 332 and a branch leg 342 extending perpendicular to the duct 332. In some embodiments, the inlet fitting 336 and/or the outlet 316 are barbed to facilitate a connection to the hose 338. In other embodiments, the inlet fitting 336 is omitted, and the gas distribution assembly 330 includes a grommet extending through the wall of the duct 332. In some such embodiments, the grommet is configured to receive and seal against the hose 338. The hose 338 extends between the inlet fitting 336 or the grommet and the outlet 316, fluidly coupling the gas distribution volume 334 and the outlet 316. The hose 338 may be made of a non-permeable material (e.g., silicone) to minimize the leakage of neutralizer out of the hose 338.

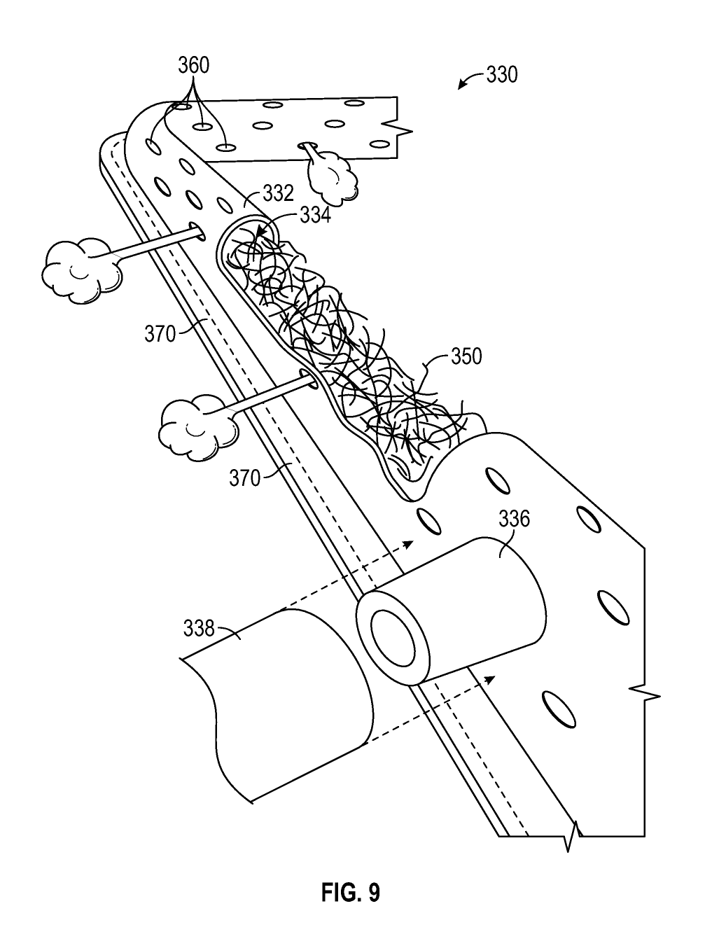

In some embodiments, the gas distribution assembly 330 is configured to resist or prevent collapsing of the duct 332. If the duct 332 were to collapse (e.g., due to the weight of an item loaded into the main volume 222, due to a fold in the fabric that forms the duct 332, etc.), the cross sectional area of the gas distribution volume 334 would decrease, restricting the flow of neutralizer through the duct 332. Referring to FIG. 9, the gas distribution assembly 330 further includes a support or support material, shown as filler 350, coupled to the duct 332 and positioned within the gas distribution assembly 330. The filler 350 is configured to impart a biasing force outward on the wall of the duct 332 to resist or prevent the duct 332 from collapsing (e.g., folding, crumpling, bending, buckling, etc.). The filler 350 is configured to minimally restrict airflow through the duct 332. As shown in FIG. 9, the filler 350 is a group of loose strips of resilient material (e.g., crumpled plastic, etc.) that are compressed within the duct 332. In other embodiments, the filler 350 is another type of loose packed, compressible material such as insulation or foam. Due to the compressed state of the filler 350, the strips of plastic push outward on the wall of the duct 332, preventing the duct 332 from collapsing. The filler 350 is packed loosely to minimally restrict the flow of neutralizer through the duct 332.

In other embodiments, the gas distribution assembly 330 instead includes a different type of support configured to resist collapse of the of the duct 332. By way of example, the support may be a woven metal or plastic hose that extends along the exterior or the interior of the duct 332, increasing the resistance of the duct 332 to collapsing. In such embodiments, the fibers that make up the hose may be woven loosely to minimally restrict the flow of neutralizer through the wall of the hose. By way of another example, the support is a spring (e.g., a compression spring) that extends along the length of the duct 332. Such a spring may be placed along the exterior or the interior of the duct 332. The spring may be sized to minimally restrict airflow through the duct 332.

In other embodiments, the duct 332 is otherwise configured to resist collapsing of the duct 332. By way of another example, the duct 332 may be made at least in part from a rigid material (e.g., plastic, metal, etc.) that resists deformation. In such an example, the rigid portions of the duct 332 may be coupled by flexible portions or other components configured to rotate (e.g., swivel fittings, etc.) such that the ducts 332 still facilitate deformation of the bag 100 (e.g., for storage).

Referring to FIGS. 6-11, the wall of the duct 332 defines a number of outlets, holes, perforations or apertures, shown as outlets 360, that fluidly couple the gas distribution volume 334 to the main volume 222. The outlets 360 are arranged along the length and/or along the circumference of the duct 332. The outlets 360 facilitate the distribution of neutralizer throughout the bag 100. The emitter 310 fills the gas distribution volume 334 with pressurized neutralizer, which flows throughout the duct 332 and out through the outlets 360. The pressure and flow rate of the neutralizer provided by the emitter 310 and the size, number, and relative locations of the outlets 360 may be varied to adjust the distribution of neutralizer throughout the bag 100. As shown in FIG. 8, the outlets 360 are uniformly spaced along the length of the duct 332 and are of a uniform size. In some embodiments, the fabric used to construct the duct 332 is permeable (e.g., through the spaces between the woven fibers of the fabric). This facilitates the flow of neutralizer through the fabric in addition to through the outlets 360. The openings in the fabric may be small enough that the amount of neutralizer that passes through the fabric is negligible when compared to the amount of neutralizer that passes through the outlets 360. In other embodiments, the duct 332 is made from a non-permeable (e.g., airtight, waterproof, etc.) material such that the neutralizer only passes out of the gas distribution volume 334 through the outlets 360.

In one embodiment, the pressure and flow rate of neutralizer from the emitter 310, the permeability of the duct 332, and/or the size, quantity, and/or placement of the outlets 360 are configured such that the duct 332 inflates when the neutralizer is supplied to the gas distribution volume 334. This inflation resists collapse of the duct 332. Providing a greater flow rate and/or pressure of neutralizer, decreasing the permeability of the duct 332, and/or decreasing the quantity and size of the outlets 360 may cause the neutralizer to impart a greater force outward on the duct 332, further resisting collapse of the duct 332.

In some embodiments, the gas distribution assembly 330 incorporates filter material. By way of a first example, the filler 350 be made partially or entirely from filter material. By way of another example, the duct 332 may be made partially or entirely from filter material. By way of yet another example, the gas distribution assembly 330 may include a lining made of filter material that covers an inner surface and/or an outer surface of the duct 332. The filter material may include activated carbon, zeolite, nano materials, or another type of filter material. The filter material may be configured to adsorb or absorb undesirable substances or organisms from neutralizer prior to the neutralizer exiting the gas distribution assembly 330. The addition of the filter material may further facilitate the neutralizing system 300 neutralizing undesirable substances or organisms.

Referring to FIGS. 5-7, 10, and 11, the duct 332 extends along the entire length of the seam 220 such that the gas distribution assembly 330 extends along the front side 118, the top side 110, and the rear side 120 of the bag 100. The seam 220 forms a continuous loop along the adjoining edges of the outer panel assembly 132 and the inner panel assembly 190. Accordingly, the duct 332 forms a continuous loop. Such a shape facilitates the distribution of neutralizer throughout the entirety of the duct 332, even if a portion of the duct 332 has been pinched shut (e.g., by the weight of an item resting on the duct 332). Additionally, locating the duct 332 along the front side 118, the top side 110, and the rear side 120 minimizes the likelihood that the full weight of an item will rest upon the duct 332, as the weight of items in the bag 100 is primarily supported by the bottom side 112 of the bag 100.

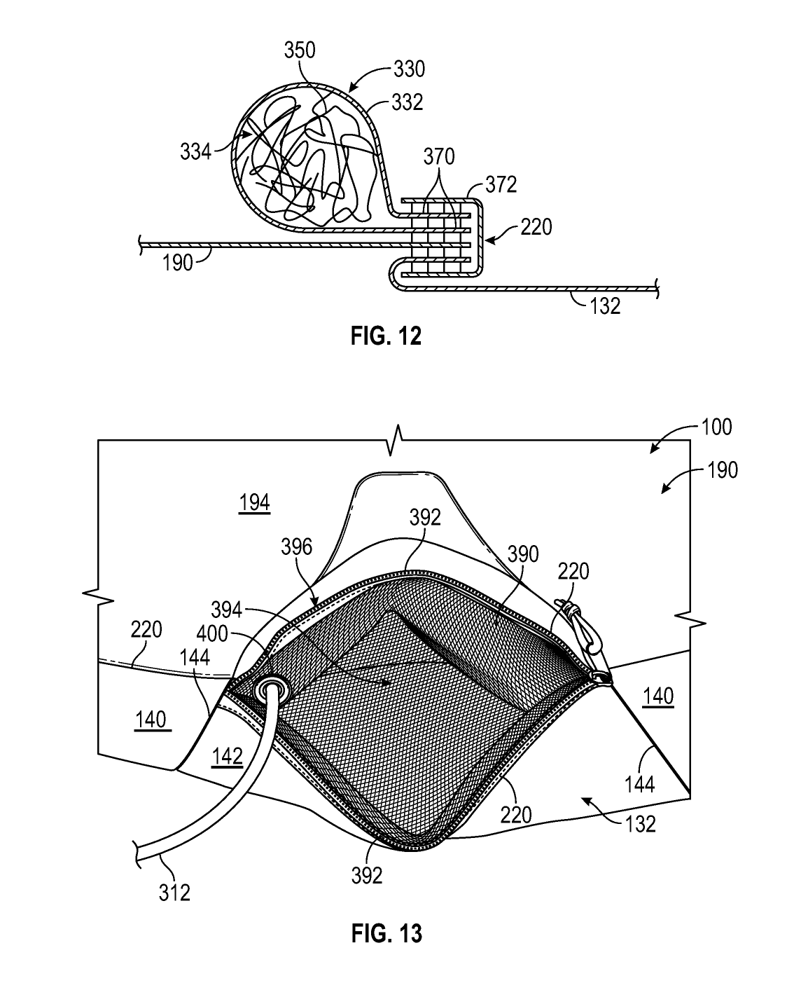

To couple the duct 332 to the outer panel assembly 132 and the inner panel assembly 190, the duct 332 is inserted into the seam 220. FIG. 12 shows a cross section of the seam 220 and the surrounding portion of the bag 100. To make the seam 220, the sheet of material used to make the duct 332 is folded over upon itself to form the gas distribution volume 334. With the duct 332 folded, both edge portions of the sheet of material used to make the duct 332 form flange portions 370 that are arranged adjacent one another and facing the same direction. The inner edge portion of the outer panel assembly 132 and the outer edge portion of the inner panel assembly 190 are arranged adjacent the flange portions 370 and facing the same direction as the flange portions 370, forming a stack containing each edge portion and flange portion 370. A cover 372 (e.g., a piece of fabric) is wrapped around the bundle along the entire length of the seam 220. Thread passes through the flange portions 370, the inner edge portion of the outer panel assembly 132, the outer edge portion of the inner panel assembly 190, and through the cover 372, stitching the various components of the seam 220 together. At various points along the length of the seam 220, additional components (e.g., the zipper 392, the walls 232 of the pockets 230 and the pockets 240, additional liners extending along one or more inner surfaces of the bag 100, etc.) can also be added to the bundle inside of the cover 372 and stitched, becoming part of the seam 220. In other embodiments, the duct 332 is coupled (e.g., sewn, adhered, etc.) to another part of the bag 100.

Referring to FIG. 6, the emitter 310 is disposed within one of the pockets 230 adjacent the inlet fitting 336 (e.g., in the lower row of pockets 230 near the lateral center of the bag 100). In some embodiments, that pocket 230 includes an elastic band 236 sized such that the elastic band 236 puts constant pressure on the emitter 310 to hold it in place. To reduce the length of the hose 338, the inlet fitting 336 may be located proximate the pocket 230 that holds the emitter 310. The hose 338 and/or the electrical cord 312 may pass through the opening 234 and above the pocket 230, or the hose 338 and/or the electrical cord 312 may pass through an opening in the pocket 230 (e.g., defined by a grommet, cut into the pocket 230, etc.). FIG. 6 shows a grommet 380 and a grommet 382 coupled the wall 232 of the pocket 230. The grommet 380 defines an aperture in the pocket 230 configured (e.g., sized) such that the electrical cord 312 can pass through the grommet 380. The grommet 382 defines an aperture in the pocket 230 configured (e.g., sized) such that the hose 338 can pass through the grommet 382.

Referring to FIGS. 6 and 13, the bag 100 further includes a bag, sack, or pocket, shown as pocket 390, coupled to the outer panel assembly 132 and the inner panel assembly 190. The portion of the seam 220 along the upper edge of the central panel 142 includes a zipper 392. The pocket 390 is sewn into the seam 220 on each side of the zipper 392 such that a first edge of the pocket 390 is fixedly coupled to the central panel 142 and a second edge of the pocket 390 is fixedly coupled to the rear panel 194. The pocket 390 defines a volume, shown as cord volume 394, configured to contain a portion of the electrical cord 312. With the zipper 392 in a closed position, the zipper 392 couples the central panel 142 to the rear panel 194 such that the cord volume 394 is enclosed by the zipper 392 and the pocket 390. When in a closed position, the zipper 392 prevents the electrical cord 312 from entering or exiting the cord volume 394. With the zipper 392 in an open position, as shown in FIG. 13, the cord volume 394 can be accessed through an opening 396, allowing removal of the electrical cord 312 (e.g., to be plugged into a power outlet).

The pocket 390 is disposed directly beneath the pocket 230 that contains the emitter 310. The pocket 390 includes a grommet 400 that extends through the pocket 390 defining an aperture that connects the cord volume 394 to the main volume 222. The grommet 400 is configured such that the electrical cord 312 can pass through the aperture defined by the grommet 400. When the bag 100 is in a storage configuration, the electrical cord 312 passes from the emitter 310, through the grommet 380 and the grommet 400, and into the cord volume 394 where a length of the electrical cord 312 is stored with the zipper 392 in the closed position. When the bag 100 is in a use, active, or neutralizing configuration, the zipper 392 is moved to the open position, and the electrical cord 312 can be removed from the cord volume 394 to connect to an electrical outlet.

The emitter 310 and the gas distribution assembly 330 cooperate to distribute neutralizer throughout the main volume 222. Referring to FIGS. 4, 10, and 11, to facilitate the neutralizer passing into the side pocket volumes 172, the pockets 170 may each include one or more grommets 410 that each define an aperture fluidly coupling the side pocket volumes 172 to the main volume 222. Such grommets 410 may additionally or alternatively be used to fluidly couple other chambers (e.g., grommets 410 in the outer panel assembly 132 configured to fluidly couple the main volume 222 to the surrounding environment). Alternatively, one or more components of the bag 100 (e.g., the pockets 170) may be made from a permeable material (e.g., a loosely woven fabric) or a material having a number of apertures extending therethrough (e.g., mesh) to facilitate the flow of neutralizer to various areas of the bag 100.

As shown in FIG. 5, in some embodiments, the neutralizing system 300 further includes a piece of adsorbent material, shown as filter 420. The filter 420 is made from a material configured to passively adsorb odor particles (e.g., collect odor particles on a surface), such as carbon, neutralizing odors. To couple the filter 420 to the bag 100, the bag 100 further includes a pocket or slot, shown as pocket 422, configured to receive the filter 420. As shown in FIG. 5, the pocket 422 is coupled to an inner surface of the top flap 198. The pocket 422 may be made from a permeable material and/or define a series of apertures to facilitate airflow to the filter 420.

With the bag 100 in a neutralizing configuration, the emitter 310 pumps neutralizer through the gas distribution assembly 330 and throughout the various volumes of the bag 100 (e.g., the main volume 222, the side pocket volumes 172, etc.). The neutralizer interacts with and neutralizes any undesirable substances or organisms throughout the bag 100. Further, the filter 420 passively adsorbs odor particles, further neutralizing odors. By way of example, the bag 100 may be filled with equipment used during exercise (e.g., hockey pads, soccer jerseys, running shorts, etc.) that absorbs sweat throughout use, producing unpleasant odors. By way of another example, the bag 100 may be filled with clothing or other equipment worn by a hunter. The bag 100 neutralizes odors in the clothing or other equipment that could alert wildlife to the presence of the hunter. In both such examples, the neutralizer may interact with sweat, with odor-causing bacteria attracted by sweat, and/or directly with odor particles, halting the production of and/or neutralizing undesirable odors. The filter 420 may further adsorb odor particles not neutralized by the neutralizer.

In some embodiments, the porosity of the duct 332 varies along the length of the duct 332. Duct 332 having a variable porosity may provide a variable resistance to neutralizer passing out of the duct 332. The porosity may change gradually along the length, or the porosity may change sharply at a certain point along the length. In some embodiments, the properties of the material used to form the duct 332 are varied to change the porosity. By way of example, the duct 332 may be or include a loosely woven material that freely facilitates neutralizer passing therethrough, thereby having a relatively high porosity, along a first portion of the length. The duct 332 may be or include a more tightly woven, airtight, and/or waterproof material that resists or prevents the passage of neutralizer therethrough, thereby having a relatively low porosity, along a second portion of the length. The thickness of the material may be varied to change the porosity. A material having a first porosity may surround or may line a material having a second porosity to vary the porosity of the duct 332. Additionally or alternatively, the duct 332 may have outlets 360 that are differentially sized and/or placed along the length of the duct 332, thereby changing the porosity thereof. By way of example, the outlets 360 may arranged more densely (e.g., placed closer together) and/or the outlets 360 may be smaller along a first length of the duct 332. The outlets 360 may be arranged less densely and/or the outlets 360 may be larger along a second length of the duct 332. The pressure of the neutralizer within the gas distribution volume 334 gradually decreases along the length of the duct 332 as the neutralizer escapes the duct 332. In some embodiments, the porosity of the duct 332 may be lesser near the inlet fitting 336 and greater farther from the inlet fitting 336. This lessens the resistance to the neutralizer passing out of the duct 332 as the pressure decreases. Accordingly, this arrangement may facilitate an even distribution of neutralizer throughout the bag 100.

The ducts 332 may be arranged in various ways. By way of example, the ducts 332 may form multiple separate paths instead of a continuous loop. In some such embodiments, the ends of the ducts 332 are sealed to prevent leakage of the neutralizer. By way of another example, the bag 100 may include multiple emitters 310 that interface with the duct 332 in multiple places or that each interface with separate ducts 332. By way of another example, the ducts 332 may be arranged in various patterns (e.g., in rows, in a “star” shape with multiple ducts 332 extending from a single point, in spirals, etc.). By way of another example, the duct 332 may extend across the center of a panel instead of along a seam between two panels.

Although the components of the bag 100 have been shown and described as being made from certain materials, it should be understood that each component can be made from a variety of different materials (e.g., mesh, canvas, vinyl, nylon, PVC coated fabric, metal, etc.). It may be advantageous to utilize non-permeable materials in certain components of the bag 100. By way of example, utilizing a non-permeable material in the components of the bag 100 that are exposed to the environment may facilitate sealing odor particles within the bag 100 and/or making the bag 100 water resistant. It may be advantageous to utilize permeable materials in certain components of the bag 100. By way of example, utilizing a permeable material in the internal components of the bag 100 may facilitate the flow of neutralizer throughout the bag 100. It may be advantageous to use flexible materials in the bag 100. By way of example, using primarily flexible materials in the bag 100 may facilitate collapsing the bag 100 for storage. Although the bag 100 is described as using zippers to couple various flaps, panels, and pockets to close various openings, it should be understood that the zippers may be replaced with other types of securing mechanisms (e.g., Velcro, buttons, snaps, corresponding hooks and loops, etc.)

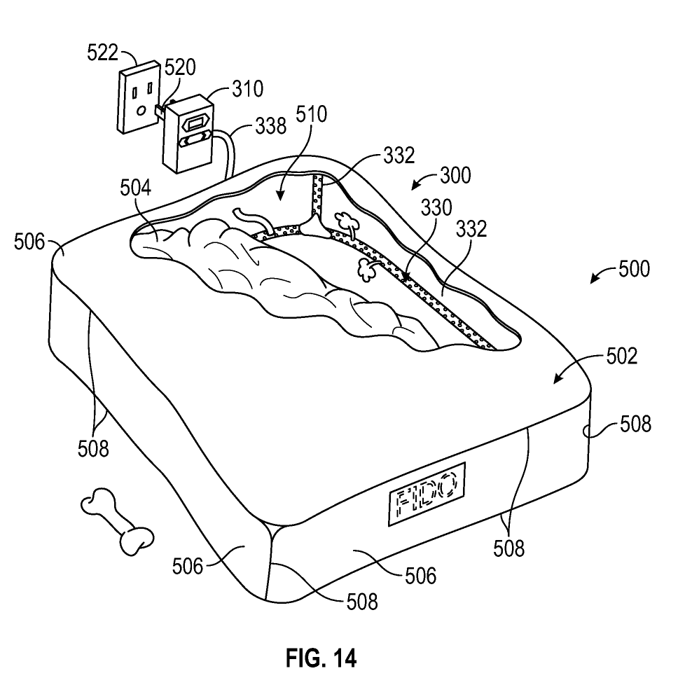

According to an alternative embodiment shown in FIG. 14, the gas distribution assembly 330 is integrated into a pet bed 500 for a dog, a cat, or another animal. The pet bed 500 includes a cover 502 (e.g., a fabric cover) filled with packing, stuffing, or fill material 504 in order to provide a comfortable surface on which animals can lay. The cover 502 includes a series of panels 506 that are coupled (e.g., sewn) together along a series of seams 508. The panels 506 define an enclosed volume 510 that contains the fill material 504. The emitter 310 may be disposed inside of or outside of the cover 502 (i.e., inside or outside of the enclosed volume 510). As shown in FIG. 14, the emitter 310 includes an electrical connector 520 that interfaces directly with an electrical outlet 522 connected to a power grid. Additionally or alternatively, the emitter 310 may include an energy storage device (e.g., positioned within the housing 324, positioned within a pocket of the cover 502, etc.).

The emitter 310 is fluidly coupled to a duct 332 by a hose 338. Alternatively, the emitter 310 may be directly fluidly coupled to a gas distribution volume of a duct 332. As shown in FIG. 14, the gas distribution assembly 330 is coupled to an inner surface of the cover 502 (e.g., extends within the enclosed volume 510) and is arranged to distribute neutralizer throughout the enclosed volume 510. The ducts 332 may be removably coupled (e.g., through attachment with a hook-and-loop fastener, etc.) or fixedly coupled (e.g., through adhesive, through being sewn into the seam 508, etc.) to the panels 506. The ducts 332 extend along the length of the seams 508 and are fluidly coupled to one another, distributing neutralizer into the enclosed volume 510. Alternatively, the ducts 332 may extend between the seams 508 and/or through the fill material 504. The ducts 332 may additionally or alternatively extend outside of the cover 502 to distribute neutralizer to the environment surrounding the pet bed 500. Further, the pet bed 500 may include one or more pockets or points of attachment for a filter 420 to facilitate adsorbing and/or absorbing odor particles. A user may operate the neutralizing system 300 to reduce the prevalence of unpleasant odors associated with pets. In other embodiments, the emitter 310 and/or the duct 332 are otherwise incorporated into the pet bed 500.

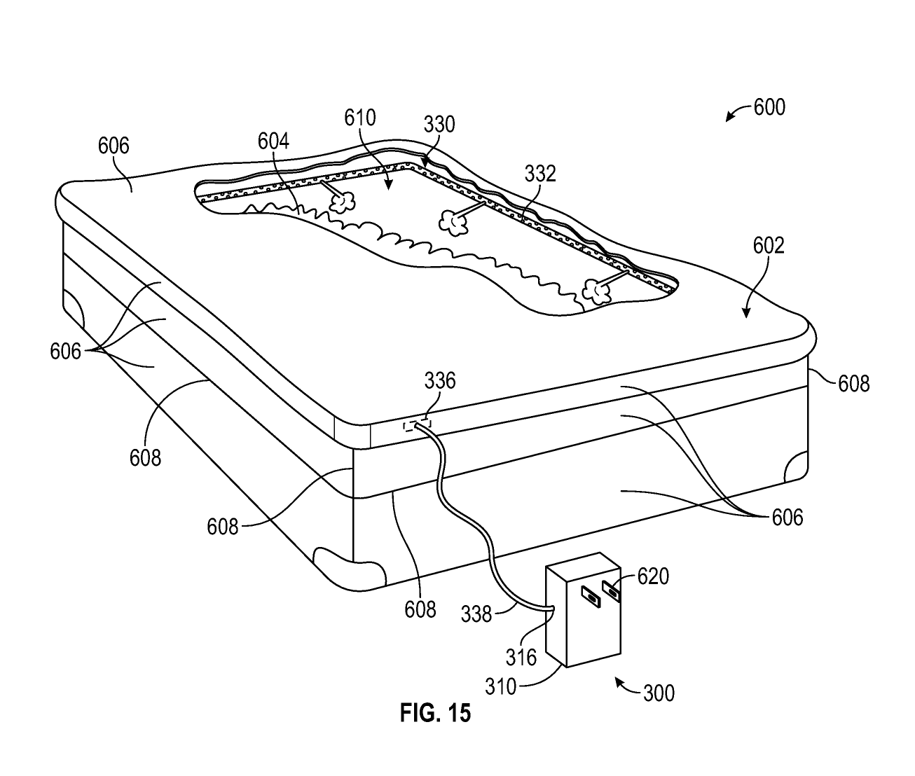

According to another alternative embodiment, shown in FIG. 15, the gas distribution assembly 330 is integrated into a mattress 600 configured to be used a human. The mattress 600 includes an enclosure, shown as cover 602 (e.g., a fabric cover), containing fill material 604 (e.g., stuffing, packing, springs, foam, etc.) that supports the weight of a user. Specifically, the cover 602 includes a series of panels 606 coupled together along a series of seams 608. The panels 606 define an enclosed volume 610 that contains the fill material 604. As shown in FIG. 15, the emitter 310 includes an electrical connector 620 that interfaces directly with an electrical outlet connected to a power grid. Additionally or alternatively, the emitter 310 may include an energy storage device (e.g., positioned within the housing 324, positioned within a pocket of the cover 602, etc.).

The emitter 310 is fluidly coupled to a gas distribution volume of a duct 332 by a hose 338. Alternatively, the emitter 310 may be directly fluidly coupled to the duct 332. As shown in FIG. 15, the gas distribution assembly 330 is coupled to an inner surface of the cover 602 (e.g., extends within the enclosed volume 610) and is arranged to distribute neutralizer throughout the enclosed volume 610. The ducts 332 may be removably coupled (e.g., through attachment with a hook-and-loop fastener, etc.) or fixedly coupled (e.g., through adhesive, through being sewn into the seam 608, etc.) to the panels 606. The ducts 332 extend along the lengths of the seams 608 and are fluidly coupled to one another, distributing neutralizer into the enclosed volume 610. Alternatively, the ducts 332 may extend between the seams 608 and/or through the fill material 604. The ducts 332 may additionally or alternatively extend outside of the cover 602 to distribute neutralizer to the environment surrounding the mattress 600. In other embodiments, the cover 602 is omitted, and the ducts 332 are directly coupled to the fill material 604. Further, the mattress 600 may include one or more pockets or points of attachment for a filter 420 to facilitate adsorbing and/or absorbing odor particles. A user may operate the neutralizing system 300 to distribute neutralizer evenly throughout the mattress 600, reducing the prevalence of any undesirable odors, substances, or organisms within the mattress 600. In other embodiments, the emitter 310 and/or the duct 332 are otherwise incorporated into the mattress 600.

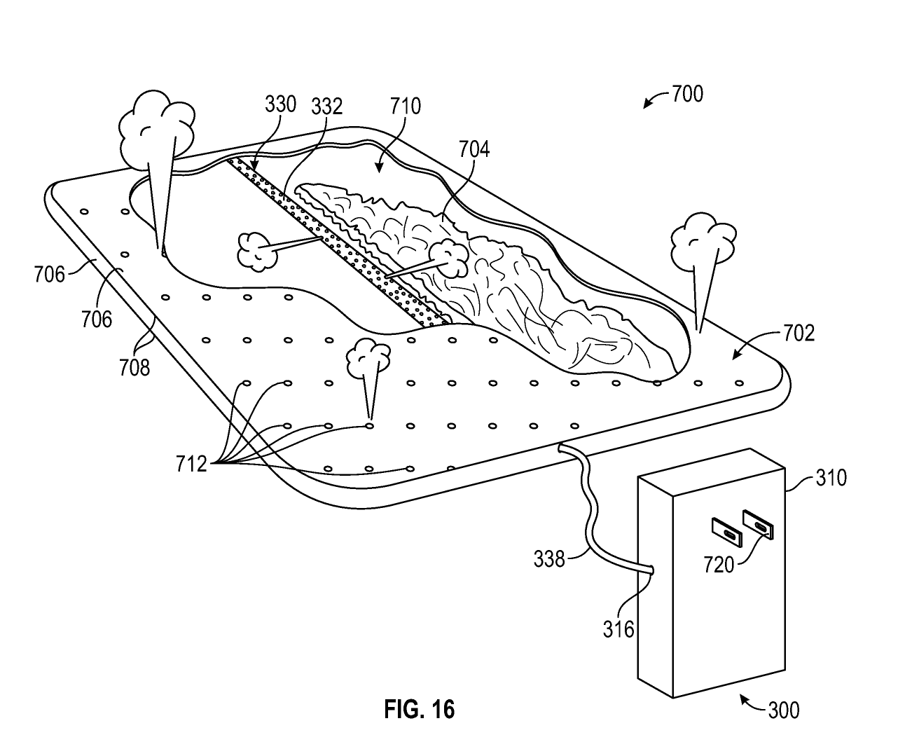

According to an alternative embodiment shown in FIG. 16, the gas distribution assembly 330 is integrated into a pad 700. The pad 700 may be a floor covering, a kennel covering, a rug, a mat, a storage container covering, or another type of pad. The pad 700 includes an enclosure or cover 702 (e.g., a fabric cover) filled with packing, stuffing, or fill material 704 configured to support the weight of an object or item resting against the pad 700. Specifically, the cover 702 includes a series of panels 706 coupled together along a series of seams 708. The panels 706 define an enclosed volume 710 that contains the fill material 704. The panels 706 define a series of apertures 712 extending from an inner surface of the cover 702 to an outer surface of the cover 702. Accordingly, the apertures 712 fluidly couple the enclosed volume 710 to the surrounding environment. As shown in FIG. 16, the emitter 310 includes an electrical connector 720 that interfaces directly with an electrical outlet connected to a power grid. Additionally or alternatively, the emitter 310 may include an energy storage device (e.g., positioned within the housing 324, positioned within a pocket of the cover 702, etc.).

The emitter 310 is fluidly coupled to a gas distribution volume of a duct 332 by a hose 338. Alternatively, the emitter 310 may be directly fluidly coupled to the duct 332. As shown in FIG. 16, the duct 332 extends along the length of and is coupled (e.g., sewn, adhered, etc.) an inner surface of the cover 702. The ducts 332 may be removably coupled (e.g., through attachment with a hook-and-loop fastener, etc.) or fixedly coupled (e.g., through adhesive, through a sewn connection, etc.) to the panels 706. Accordingly, the gas distribution assembly 330 distributes neutralizer throughout the enclosed volume 710. The apertures 712 facilitate distributing neutralizer from the enclosed volume 710 to the surrounding environment. Alternatively, the pad 700 may include one or more ducts 332 that extend within the seams 708. Further, the pad 700 may include one or more pockets or points of attachment for a filter 420 to facilitate adsorbing and/or absorbing odor particles. A user may operate the neutralizing system 300 to distribute neutralizer around an item placed against the pad 700, reducing the prevalence of odors emitted by the item. Alternatively, a user may operate the neutralizing system 300 to distribute neutralizer within a volume containing the pad 700. In other embodiments, the emitter 310 and/or the duct 332 are otherwise incorporated into the pad 700

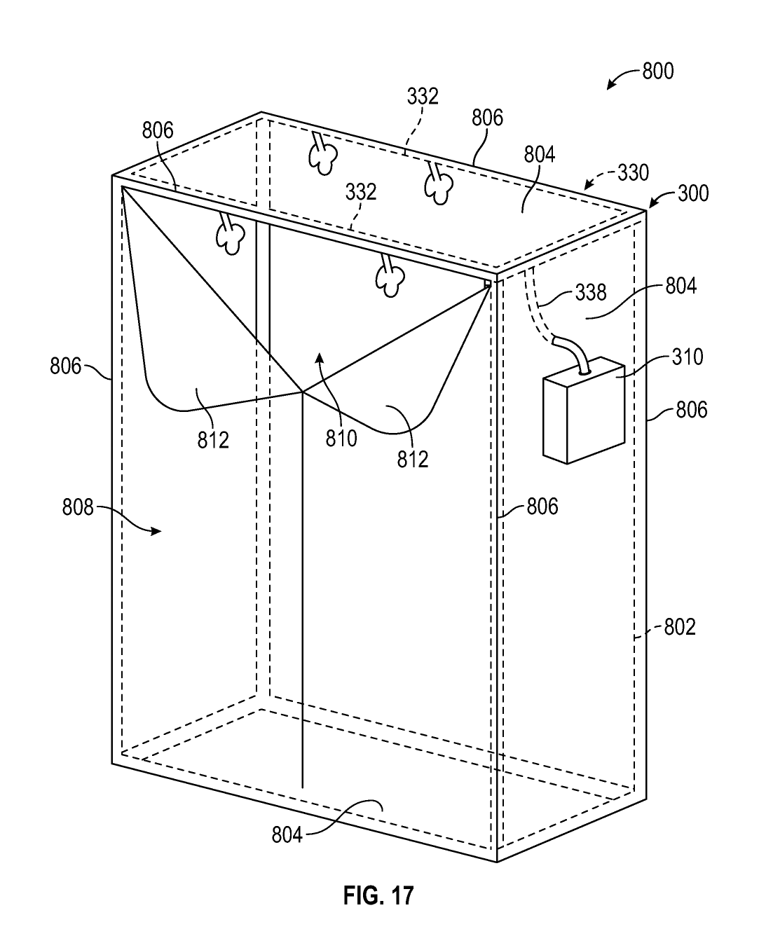

According to an alternative embodiment shown in FIG. 17, the gas distribution assembly 330 is integrated into an enclosure, shown as closet 800 (e.g., a portable closet, a closet built into a home, for use in a laundry room, for storing hunting gear, etc.). The closet 800 includes a frame 802 (e.g., including bars for hanging clothes, including shelves, etc.) configured to support clothing and equipment. The closet 800 further includes a plurality of panels 804 coupled (e.g., sewn, adhered, etc.) to one another at seams 806 to form a cover 808 that extends around the frame 802. The cover 808 defines an enclosed volume 810. One of the panels 804 includes a pair of door portions 812 that can be selectively opened to facilitate fluid communication between the enclosed volume 810 and the surrounding environment and/or facilitate access to the enclosed volume 810. With the door portions 812 closed (e.g., using zippers, etc.), the enclosed volume 810 is partially or completely sealed by the cover 808.

When used with the closet 800, the emitter 310 may include an electrical cord 312 to electrically couple to an external source of electrical energy (e.g., a generator, a power outlet coupled to a power grid, a power outlet coupled to an alternator of a vehicle, etc.). Additionally or alternatively, the emitter 310 may include an energy storage device (e.g., positioned within the housing 324, positioned within a pocket of the cover 808, etc.). The gas distribution assembly 330 is disposed within the enclosed volume 810. A gas distribution volume of a duct 332 is fluidly coupled to the emitter 310 (e.g. with a hose 338, directly, etc.) to distribute neutralizer to clothing and/or equipment contained within the enclosed volume 10. The ducts 332 may be removably coupled (e.g., through attachment with a hook-and-loop fastener, etc.) or fixedly coupled (e.g., through adhesive, through a sewn connection, etc.) to the cover 808. By way of example, the ducts 332 may be inserted into the seams 806 and sewn in place. Further, the frame 802 or the cover 808 may include one or more pockets or points of attachment for a filter 420 to facilitate adsorbing and/or absorbing odor particles within the enclosed volume 810. In other embodiments, the emitter 310 and/or the duct 332 are otherwise incorporated into the closet 800.

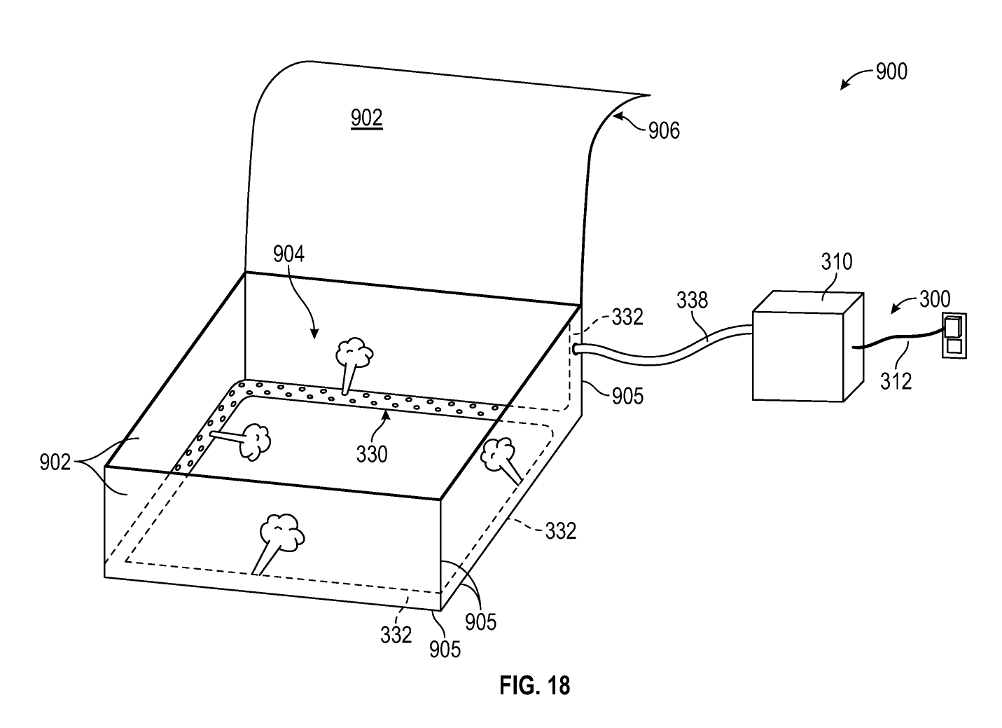

According to another embodiment shown in FIG. 18, the gas distribution assembly 330 is integrated into a container or enclosure, shown as bag 900. The bag 900 includes a series of panels 902 that define an enclosed volume 904 of the bag 900. The panels 902 are coupled (e.g., sewn, adhered, etc.) to one another along seams 905. At least one of the panels 902 includes a door or lid portion 906 that is selectively coupled to at least one of (a) itself and (b) the other panels 902 (e.g., with a zipper). The lid portion 906 may be opened to facilitate access to the enclosed volume 904 or closed to at least partially seal the enclosed volume 904 from the surrounding atmosphere. The panels 902 may be sized to facilitate the bag 900 containing a variety of different items within the enclosed volume 904. By way of example, the bag 900 may be sized to receive a mattress (e.g., a twin, double, queen, or king size mattress), a dog bed, or one or more pieces of equipment (e.g., a set of football pads, police or military protective gear, a pair of boots, etc.).

The emitter 310 may be coupled to the bag 900. By way of example, the bag 900 may include a pocket coupled to one or more of the panels 902 that receives the emitter 310. Alternatively, the emitter 310 may be placed away from the bag 900. When used with the bag 900, the emitter 310 may include an electrical cord 312 to electrically couple to an external source of electrical energy (e.g., a generator, a power outlet coupled to an alternator of a vehicle, etc.). Additionally or alternatively, the emitter 310 may include an energy storage device (e.g., positioned within the housing 324, positioned within a pocket of the bag 900, etc.).

The bag 900 further includes the gas distribution assembly 330. The ducts 332 may be removably coupled (e.g., through attachment with a hook-and-loop fastener, etc.) or fixedly coupled (e.g., through adhesive, through being sewn into the seams 905, etc.) to the panels 902. The gas distribution volume of the gas distribution assembly 330 is fluidly coupled to the emitter 310 (e.g., directly, indirectly through a hose 338, etc.). The ducts 332 may extend along one of the seams 905 or along a surface of one of the panels 902. As shown in FIG. 18, the ducts 332 extend along the seams 905. Each of the ducts 332 extend within the enclosed volume 904. Accordingly, the ducts 332 distribute neutralizer within the enclosed volume 904. Alternatively, the gas distribution assembly 330 may be omitted, and a hose 338 may supply neutralizer directly to the enclosed volume 904. The neutralizing system 300 may be used to reduce the prevalence of odors of items positioned within the enclosed volume 904. By way of example, a user may place items within the enclosed volume 904, close the lid portion 906, and turn on the neutralizing system 300. In other embodiments, the emitter 310 and/or the duct 332 are otherwise incorporated into the bag 900.

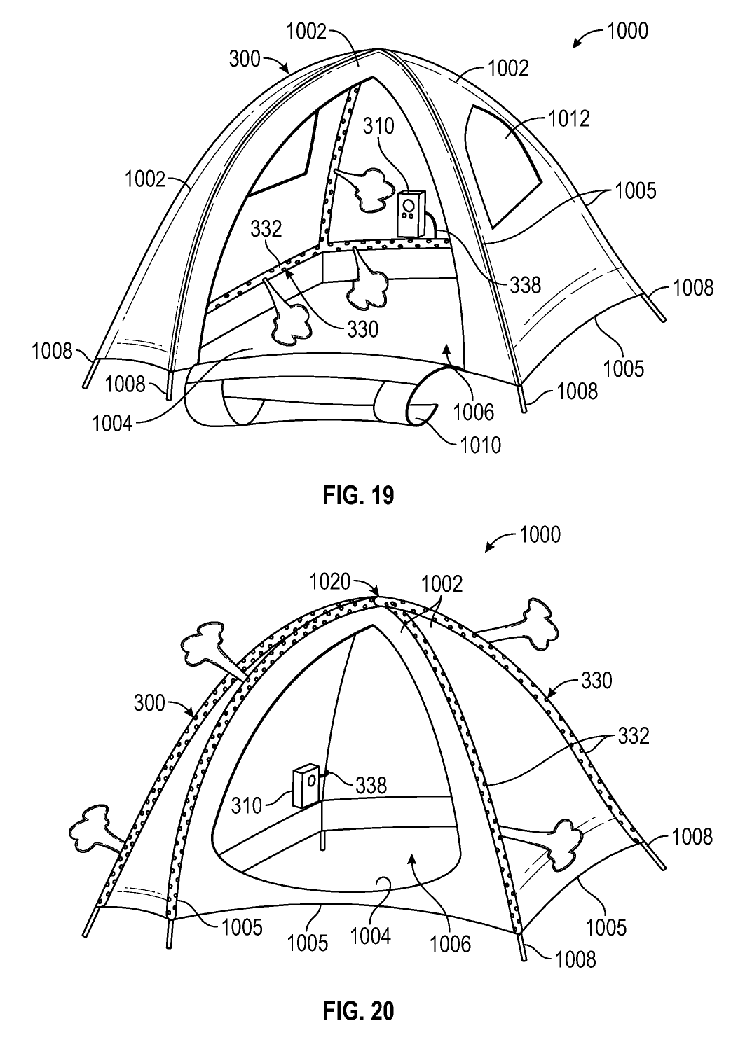

According to another embodiment shown in FIGS. 19-21, the gas distribution assembly 330 is integrated into an enclosure, shown as hunting blind or tent 1000. The tent 1000 includes a series of panels, shown as wall panels 1002 and floor panel 1004. The wall panels 1002 are coupled to one another with seams 1005 along vertically-extending edges, and each wall panel 1002 is coupled to the floor panel 1004 with a seam 1005 along a bottom edge, defining an enclosed volume 1006. A series of support members, shown as rods 1008, extend along the seams 1005 between the wall panels 1002, providing support to hold the wall panels 1002 above the floor panel 1004. One or more of the wall panels 1002 include a door portion 1010 that can be selectively opened to facilitate fluid communication between the enclosed volume 1006 and the surrounding environment and/or to facilitate access to the enclosed volume 1006. In some embodiments, some of the wall panels 1002 include window portions 1012 that can be selectively opened to facilitate fluid communication between the enclosed volume 1006 and the surrounding environment and/or to facilitate access to the enclosed volume 1006. In some embodiments, the tent 1000 is a sleeping enclosure that is used to house one or more users (e.g., while camping). In some embodiments, the tent 1000 is a hunting blind configured to camouflage users while hunting. In some embodiments, the tent 1000 is an enclosure used to reduce the prevalence of the odors of items placed therein.

The tent 1000 is coupled to the emitter 310. By way of example, the tent 1000 may include a pocket coupled to one of the wall panels 1002 that receives the emitter 310. When used with the tent 1000, the emitter 310 may include an energy storage device internally (e.g., inside of the housing 324, etc.), or the tent 1000 may include one or more additional pockets that support energy storage devices. Additionally or alternatively, the emitter 310 may include an electrical cord 312 to electrically couple to an external source of electrical energy (e.g., a generator, a power outlet coupled to an electrical grid, etc.).

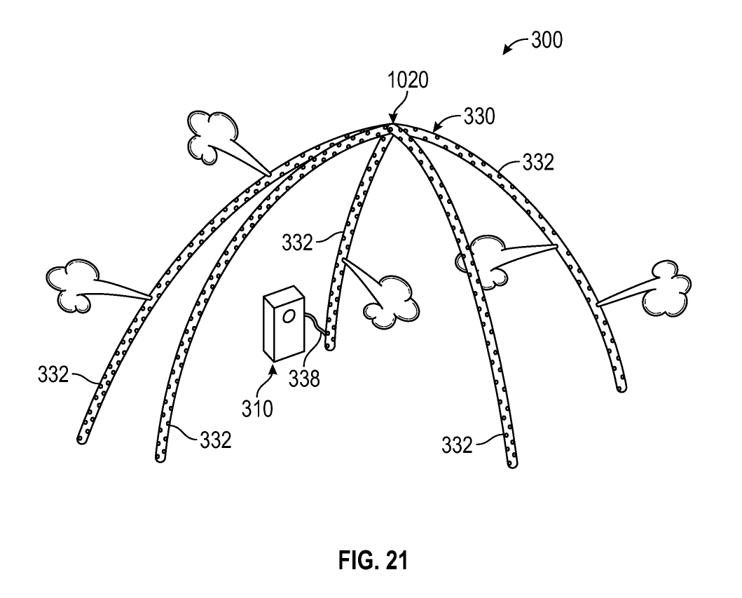

The tent 1000 further includes the gas distribution assembly 330. The ducts 332 may be removably coupled (e.g., through attachment with a hook-and-loop fastener, etc.) or fixedly coupled (e.g., through adhesive, through being sewn into the seams 1005, etc.) to the wall panels 1002. A gas distribution volume of the gas distribution assembly 330 is fluidly coupled to the emitter 310 (e.g., directly, indirectly through a hose 338, etc.). The ducts 332 may extend along the seams 1005, along an exterior surface of the wall panels 1002 and/or the floor panel 1004, and/or along an interior surface of the wall panels 1002 and/or the floor panel 1004. As shown in FIG. 19, the ducts 332 extend vertically within and along the seams 1005 between the wall panels 1002 and laterally between the seams 1005. Each of the ducts 332 extend within the enclosed volume 1006. Accordingly, the ducts 332 distribute neutralizer within the enclosed volume 1006. In this embodiment, the neutralizing system 300 may be used to neutralize odors of items positioned within the enclosed volume 1006. By way of example, user may place items within the enclosed volume 1006, close the door portion 1010 and window portions 1012, and turn on the neutralizing system 300. In an alternative embodiment shown in FIGS. 20 and 21, the ducts 332 extend along the seams 1005 between the wall panels 1002 and are all fluidly coupled at an intersection point 1020 at the top of the tent 1000. Each of the ducts 332 extend along an exterior surface of the tent 1000. Accordingly, the ducts 332 distribute neutralizer into the surrounding environment outside of the enclosed volume 1006. In this embodiment, the neutralizing system 300 may be used to neutralize odors emitted by one or more users within the enclosed volume 1006 (e.g., to prevent detection by a target species). Further, the tent 1000 may include one or more pockets or points of attachment for a filter 420 to facilitate adsorbing and/or absorbing odor particles. In other embodiments, the emitter 310 and/or the duct 332 are otherwise incorporated into the tent 1000.

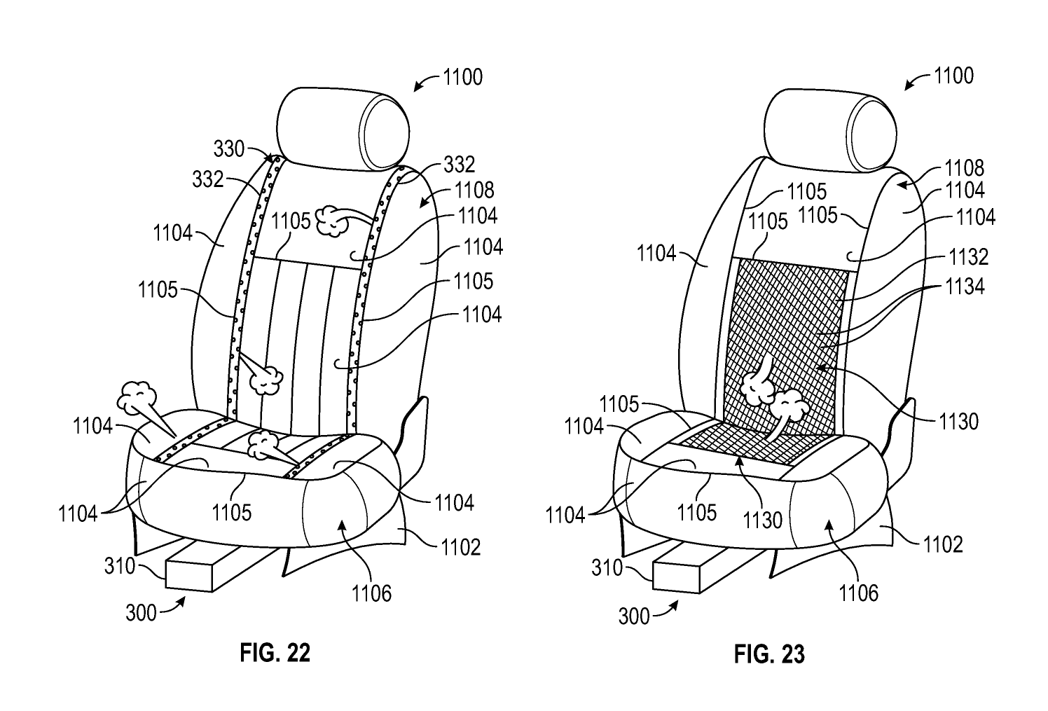

According to other embodiments shown in FIGS. 22 and 23, the gas distribution assembly 330 is integrated into an upholstery or covering, shown as seat cover 1100, which contacts a user sitting in a seat 1102. The seat 1102 may be a seat of a car, a dining room chair, a barstool, a recliner, a booth chair, or any other type of seat. The seat cover 1100 may be removable or permanently attached to the seat 1102 (e.g., part of a seat assembly). The seat cover 1100 includes a series of panels 1104 (e.g., fabric panels, etc.) coupled (e.g., sewn, adhered, etc.) together along seams 1105. The panels 1104 define a bottom cover 1106, which supports a user's bottom, and a back cover 1108, which supports a user's back. The seat cover 1100 fits tightly to the structure of the seat 1102 and may have a pleasant and/or gripping exterior texture.

The emitter 310 is coupled to the seat 1102. By way of example, the seat cover 1100 may include a pocket coupled to one or more of the panels 1104 that receives the emitter 310. Alternatively, the emitter 310 may be set on the floor beneath the seat 1102. When used with the seat cover 1100, the emitter 310 may include an electrical cord 312 to electrically couple to an external source of electrical energy (e.g., a generator, a power outlet coupled to an alternator of a vehicle, etc.). Additionally or alternatively, the emitter 310 may include an energy storage device (e.g., positioned within the housing 324, positioned within a pocket of the seat cover 1100, etc.).

As shown in FIG. 22, the seat cover 1100 further includes the gas distribution assembly 330. The ducts 332 may be removably coupled (e.g., through attachment with a hook-and-loop fastener, etc.) or fixedly coupled (e.g., through adhesive, through being sewn into the seams 1105, etc.) to the panels 1104. A gas distribution volume of the gas distribution assembly 330 is fluidly coupled to the emitter 310 (e.g., directly, indirectly through a hose 338, etc.). The ducts 332 extend along the seams 1105 between the panels 1104 positioned along the bottom and back of the user. Accordingly, the ducts 332 distribute neutralizer along the surfaces of the seat cover 1100 that contact the user on a regular basis. In other embodiments, the ducts 332 extend within one or more enclosed volumes defined by the seat cover 1100. Further, the seat cover 1100 may include one or more pockets or points of attachment for a filter 420 to facilitate adsorbing and/or absorbing odor particles.

According to an alternative embodiment, shown in FIG. 23, the seat cover 1100 includes a pair of gas distribution pads, shown as pads 1130, configured to distribute neutralizer over an area. In some embodiments, the pads 1130 are similar in construction to the pad 700. Accordingly, each pad may include a cover 1132 (e.g., a fabric cover) filled with packing, stuffing, or fill material configured to support the weight of an object resting against the pad 1130. The gas distribution assembly 330 may be coupled (e.g., sewn, adhered, with a hook-and-loop fastener, etc.) to an inner surface or an outer surface of the cover 1132 and arranged to distribute neutralizer throughout an enclosed volume defined by the cover 1132. Alternatively, the gas distribution assembly 330 may be omitted, and the neutralizer may be supplied directly to the enclosed volume. The cover 1132 defines a series of apertures 1134 extending from an inner surface (e.g., a surface adjacent the enclosed volume) to an outer surface of the cover 1132. The apertures 1134 facilitate distributing neutralizer along the exterior surface of the pad 1130. Further, the seat cover 1100 may include one or more pockets or points of attachment for a filter 420 to facilitate adsorbing and/or absorbing odor particles. In other embodiments, the emitter 310 and/or the duct 332 are otherwise incorporated into the seat cover 1100.

In each embodiment, the neutralizing system 300 may be operated to reduce the prevalence of odors and odor causing substances within the seat cover 1100, the seat 1102, and the surrounding area (e.g., the cabin of a vehicle). By way of example, the neutralizing system 300 may be used to neutralize unpleasant odors (e.g., from smoking, from substances produced by animals or humans, etc.) that build up on fabric surfaces of a vehicle over time. By way of another example, the neutralizing system 300 may be used to neutralize unpleasant odors emitted from an object that is placed onto the seat cover 1100 (e.g., clothing, equipment, etc.).

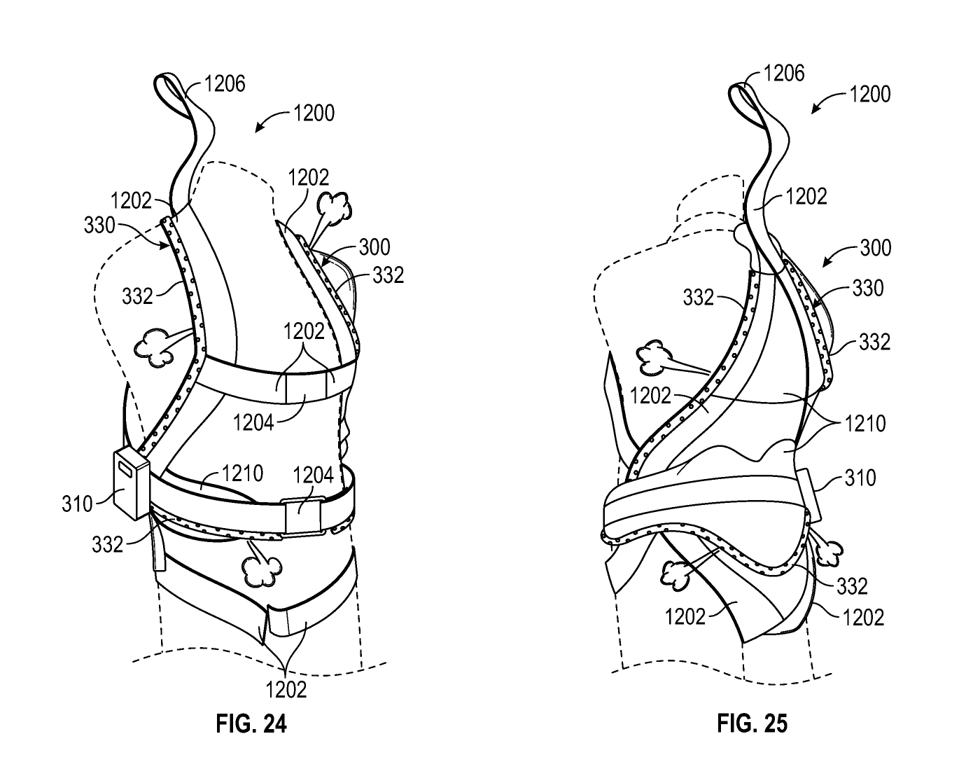

According to another embodiment shown in FIGS. 24 and 25, the gas distribution assembly 330 is integrated into a support apparatus or wearable device, shown as harness 1200. The harness 1200 includes a series of supports, shown as straps 1202, that extend around the body (e.g., the torso and thigh areas) of a user. The straps 1202 form a series of apertures, through which the user extends their arms and legs. The harness 1200 further includes one or more connectors, shown as latches 1204, that selectively couple one or more of the straps 1202 together to form one or more loops. With the latches 1204 in a connected configuration, the straps 1202 and latches 1204 prevent a user from exiting the harness 1200. One or more of the straps 1202 extend away from the body of the user and form an attachment point, shown as anchor point 1206. The anchor point 1206 forms a loop configured to couple the harness 1200 to one or more ropes, cables, straps, or chains (e.g., through a carabiner). The harness 1200 further includes a series of sheets or pads 1210 coupled to the straps 1202 that are wider than the straps 1202. The harness 1200 is configured to support the weight of the user continuously and/or during a fall event. By way of example, the harness 1200 may be used to suspend a user continuously at a desired height (e.g., when working on the exterior of a structure). By way of another example, the harness 1200 may be a safety harness for precautionary use on an elevated platform, such as a tree stand or a crane. The pads 1210 may facilitate distributing the weight of the user over a wider area, improving the strength of the harness 1200 and user comfort.

The emitter 310 is coupled to the harness 1200. By way of example, the harness 1200 may include a pocket coupled to one of the straps 1202 that receives the emitter 310. When used with the harness 1200, the emitter 310 may include an energy storage device internally (e.g., inside of the housing 324, etc.), or the harness 1200 may include one or more additional pockets that support energy storage devices. By incorporating the energy storage device into the harness 1200 and/or the emitter 310, the harness 1200 may be used away from an external source of electrical energy (e.g., an electrical outlet). Additionally or alternatively, the emitter 310 may include an electrical cord 312 to electrically couple to an external source of electrical energy (e.g., a generator, a power outlet coupled to an electrical grid, etc.).

The harness 1200 further includes the gas distribution assembly 330. The ducts 332 may be removably coupled (e.g., through attachment with a hook-and-loop fastener, etc.) or fixedly coupled (e.g., through adhesive, through a sewn connection, etc.) to the straps 1202 and/or the pads 1210. A gas distribution volume of the gas distribution assembly 330 is fluidly coupled to the emitter 310 (e.g., directly, indirectly through a hose 338, etc.). As shown in FIG. 24, the emitter 310 is positioned near waist height, and the ducts 332 extend away from the emitter 310 along the edges of certain straps 1202. Accordingly, the ducts 332 distribute neutralizer around the entire harness 1200. The ducts 332 may be coupled to exterior or interior surfaces of the straps 1202 and/or pads 1210. By way of example, the straps 1202 may be formed from two sheets of material, and a duct 332 may extend between the two sheets of material. By way of another example, a duct 332 may extend between a strap 1202 and a pad 1210. Further, the harness 1200 may include one or more pockets or points of attachment for a filter 420 to facilitate adsorbing and/or absorbing odor particles. In other embodiments, the emitter 310 and/or the duct 332 are otherwise incorporated into the harness 1200.

The neutralizing system 300 may be operated while the harness 1200 is worn by the user or after the harness 1200 is removed. By way of example, the neutralizing system 300 may be used to neutralize odors produced by a hunter during a hunt to prevent detection of the hunter by a target species. By way of another example, the neutralizing system 300 may be used to reduce the prevalence of odors while the harness 1200 is in storage.

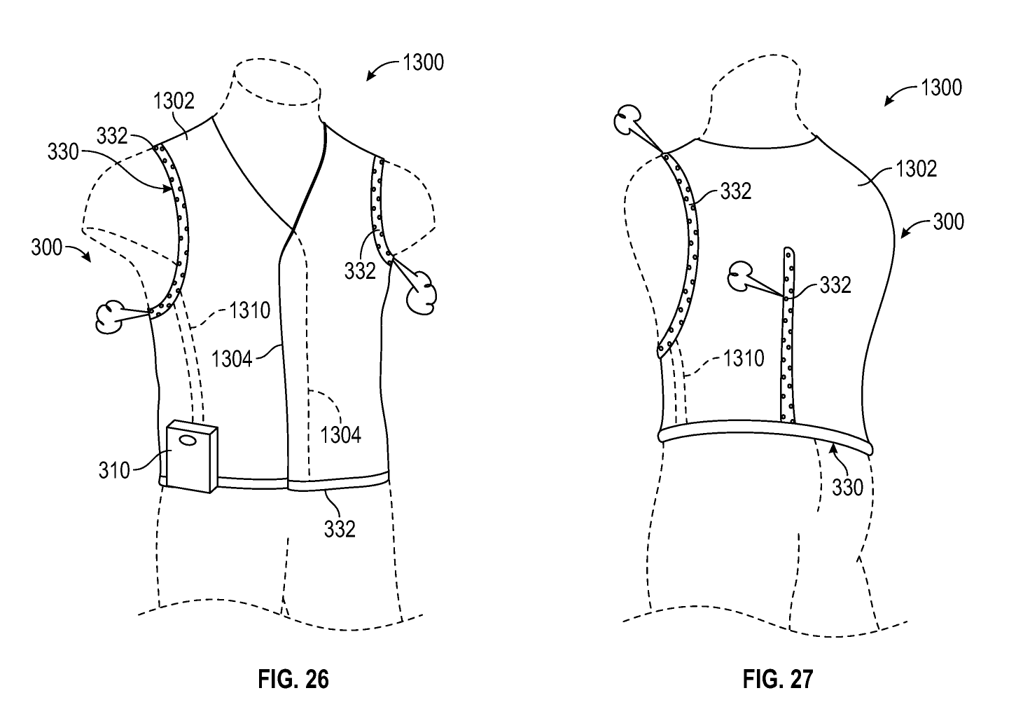

According to another embodiment shown in FIGS. 26 and 27, the gas distribution assembly 330 is integrated into a piece of clothing or wearable device, shown as vest 1300. The vest 1300 is configured to be supported by the torso of a user. The vest 1300 includes one or more sheets of material or panels that form a main body 1302 of the vest 1300. The main body 1302 defines apertures through which the arms, waist, and neck of a user extend. As shown in FIG. 26, the main body 1302 includes a pair of front portions 1304 that are selectively coupled to one another (e.g., with a zipper or snaps, etc.) to facilitate entering or exiting the vest 1300. Alternatively, the front portion 1304 may be formed from a continuous piece of material. In some embodiments, the main body 1302 is configured to insulate the user to retain body heat (e.g., includes multiple layers of material, includes insulative material such as down or polyester fiberfill, etc.). In other embodiments, the main body 1302 is configured to facilitate a transfer of body heat to the surrounding environment (e.g., includes a thin material, includes a mesh material, etc.). The main body 1302 may be configured specifically for minimal visibility (e.g., camouflage, etc.) or high visibility (e.g., a bright color such as orange or yellow). In other embodiments, the vest 1300 is instead a different type of clothing, such as a shirt or jacket.

The vest 1300 is coupled to the emitter 310. By way of example, the vest 1300 may include a pocket coupled to the main body 1302 that receives the emitter 310. When used with the vest 1300, the emitter 310 may include an energy storage device positioned internally (e.g., inside of the housing 324, etc.), or the vest 1300 may include one or more additional pockets that support energy storage devices. By incorporating the energy storage device into the vest 1300 and/or the emitter 310, the vest 1300 may be used away from an external source of electrical energy (e.g., an electrical outlet). Additionally or alternatively, the emitter 310 may include an electrical cord 312 to electrically couple to an external source of electrical energy (e.g., a generator, a power outlet coupled to an electrical grid, etc.).

The vest 1300 further includes the gas distribution assembly 330. The ducts 332 may be removably coupled (e.g., through attachment with a hook-and-loop fastener, etc.) or fixedly coupled (e.g., through adhesive, through a sewn connection, etc.) to the main body 1302. A gas distribution volume of the gas distribution assembly 330 is fluidly coupled to the emitter 310 (e.g., directly, indirectly through a hose 338, etc.). As shown in FIGS. 26 and 27, the emitter 310 is positioned near waist height, and the ducts 332 extend along the edges and the back of the main body 1302 away from the emitter 310. Accordingly, the ducts 332 distribute neutralizer around the entire vest 1300. Conduits, ducts, or hoses, shown as coupling portions 1310, fluidly couple a portion of the gas distribution assembly 330 extending around the waist of the user to portions of the gas distribution assembly 330 extending around the arms of the user. The coupling portions 1310 extend within the main body 1302 and accordingly may not be visible from the exterior of the vest 1300. The coupling portions 1310 may include apertures to distribute neutralizer similar to the ducts 332 or may not be permeable. The ducts 332 may extend along an edge of the main body 1302, along an exterior surface of the main body 1302, and/or within the main body 1302 (e.g., between layers of material that make up the main body 1302). By way of example, the main body 1302 may be formed from two sheets of material, and a duct 332 may extend between the two sheets of material. By way of another example, a duct 332 may extend along an outer surface of the main body 1302. Further, the vest 1300 may include one or more pockets or points of attachment for a filter 420 to facilitate adsorbing and/or absorbing odor particles.

The neutralizing system 300 may be operated while the vest 1300 is worn by the user or after the vest 1300 is removed. By way of example, the neutralizing system 300 may be used to neutralize odors produced by a hunter during a hunt to prevent detection of the hunter by a target species. By way of another example, the neutralizing system 300 may be used to neutralize odors while the vest 1300 is in storage.

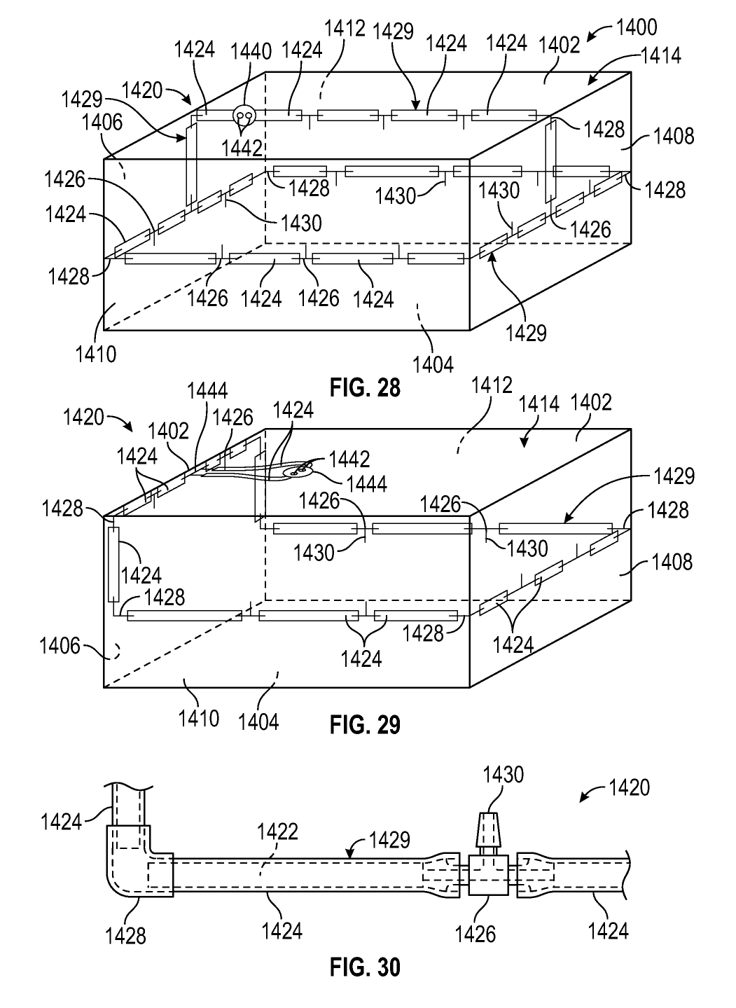

Referring to FIGS. 28 and 29, an enclosure 1400 (e.g., a bag, a closet, a tent, a mattress, a pad, etc.) is shown according to various exemplary embodiments. In one embodiment, the enclosure 1400 includes a series of panels or walls, shown as top panel 1402, bottom panel 1404, left panel 1406, right panel 1408, front panel 1410, and rear panel 1412. The panels are coupled to one another to form the enclosure 1400. The top panel 1402, the bottom panel 1404, the left panel 1406, the right panel 1408, the front panel 1410, and the rear panel 1412 define a top side, a bottom side, a left side, a right side, a front side, and a rear side of the enclosure 1400, respectively. The panels define a volume, shown as enclosed volume 1414, configured to receive and contain one or more items.

Referring to FIGS. 28-30, the enclosure 1400 includes a gas distribution assembly 1420 configured to distribute neutralizer throughout the enclosure 1400. The gas distribution assembly 1420 may be substantially similar to the gas distribution assembly 330 except as otherwise specified herein. The gas distribution assembly 1420 can be used in place of the gas distribution assembly 330 in any of the other embodiments disclosed herein (e.g., in the bag 100, in the in the pet bed 500, with the harness 1200, with the vest 1300, etc.).

The gas distribution assembly 1420 defines a gas distribution volume 1422 configured to be fluidily coupled to an embitter (e.g., the emitter 310) to receive a supply of neutralizer. Specifically, the gas distribution assembly 1420 includes a series of hoses, conduits, or tubes, shown as tubes 1424, coupled to a series of fittings, shown as tee fittings 1426 and elbow fittings 1428. As shown in FIG. 30, each tee fitting 1426 includes a pair of legs that are each inserted into an end of an adjacent tube 1424, and each angled fitting receives an end of an adjacent tube 1424. In this way, the tee fittings 1426 and the elbow fittings 1428 fluidly couple the adjacent tubes 1424. In other embodiments, the tee fittings 1426 receive the ends of adjacent tubes 1424 and/or the angled fittings are inserted into the ends of adjacent tubes 1424. Together, the tubes 1424, the tee fittings 1426, and the elbow fittings 1428 form a tube, duct, or conduit, shown as conduit 1429, that defines the gas distribution volume 1422. Each tee fitting 1426 includes a third leg defining an aperture, shown as outlet 1430, fluidly coupled to the gas distribution volume 1422. One or more of the tee fittings 1426 are arranged without a tube 1424 coupled to the third leg. These outlets 1430 are positioned within the enclosed volume 1414 such that the outlets 1430 fluidly couple the gas distribution volume 1422 with the enclosed volume 1414.

Referring to FIGS. 28 and 29, the gas distribution assembly 1420 includes a connector, shown as pass-through 1440. The pass-through 1440 defines a pair of apertures or passages, shown as ports 1442, each fluidly coupled to one of the tubes 1424. The pass-through 1440 is coupled to the top panel 1402 and extends outside of the enclosed volume 1414 such that the ports 1442 are accessible from outside of the enclosure 1400. The ports 1442 are configured to be fluidly coupled to an emitter (e.g., the emitter 310) such that neutralizer from the emitter enters the gas distribution volume 1422 through the ports 1442. By way of example, the emitter may include two outlets (e.g., outlets 316), each fluidly coupled to one of the ports 1442. By way of another example, the emitter may include one outlet, and a splitter (e.g., a tee fitting 1426) may be positioned between the emitter and the pass-through to distribute the neutralizer to both of the ports 1442. The pass-through 1440 may facilitate selectively coupling the emitter to the gas distribution assembly 1420 such that the emitter can be quickly and easily detached when not in use without having to remove the gas distribution assembly 1420 from the enclosure 1400.

FIG. 28 illustrates a first arrangement of the gas distribution assembly 1420. In FIG. 28, the pass-through 1440 is positioned on the top panel 1402. One port 1442 is fluidly coupled to a first conduit 1429, and the other port 1442 is fluidly coupled to a second conduit 1429. The first and second conduits 1429 extend in opposite directions, away from the pass-through 1440 and along the top panel 1402. The first and second conduits 1429 reach the left panel 1406 and the right panel 1408, respectively, and extend downward along each respective panel. A third conduit 1429 forms a continuous loop along the front panel 1410, the left panel 1406, the rear panel 1412, and the right panel 1408. The first and second conduits 1429 fluidly couple to the third conduit through a pair of tee fittings 1426.

FIG. 29 illustrates a second arrangement of the gas distribution assembly 1420. In FIG. 29, the pass-through 1440 is positioned on the top panel 1402. A conduit 1429 extends along the top panel 1402 and the left panel 1406. The conduit 1429 includes a fitting, shown as double tee fitting 1444. The double tee fitting 1444 includes two legs, each of which connect to a tube 1424 of the conduit 1429. The double tee fitting 1444 further includes another pair of legs, each of which is fluidly coupled to one of the ports 1442 of the pass-through 1440 through a tube 1424. The conduit 1429 extends to the front panel 1410 and the rear panel 1412, then extends downwards. The conduit 1429 then extends along the front panel 1410, the right panel 1408, and the rear panel 1412 to form a continuous loop. In other embodiments, the conduits 1429 are otherwise arranged. By way of example, the gas distribution assembly 1420 may include one or more conduits 1429 that extend along one or more of the panels without forming a closed loop.

The connections between the tubes 1424, the tee fittings 1426, and the elbow fittings 1428, are substantially sealed. In some such embodiments, the tubes 1424 are substantially airtight (e.g., made from an airtight material such as silicone, coated in an airtight material such as a waterproof membrane, etc.). This prevents transfer of neutralizer from the gas distribution volume 1422 to the enclosed volume 1414 except through the outlets 1430. This facilitates control over the pressure drop throughout the gas distribution assembly 1420. In this way, the gas distribution assembly 1420 can be configured such that the neutralizer is evenly distributed throughout the enclosed volume 1414 instead of concentrated nearest the connection to the emitter 310. In some embodiments, the conduits 1429 include at least one outlet 1430 per foot of conduit 1429. In on embodiments, the outlets 1430 are placed approximately 6 inches apart from one another.

In some embodiments, the tubes 1424 are made from a flexible material (e.g., fabric coated in a waterproof membrane, silicone, etc.). This facilitates routing the gas distribution assembly 1420 around obstacles and/or corners. In embodiments where one or more of the panels of the enclosure 1400 are flexible, the tubes 1424 deform to facilitate deformation of the panels. Such deformation may occur when packing items into the enclosure 1400 or when collapsing the enclosure 1400 for storage.

The gas distribution assembly 1420 may be configured to resist or prevent collapsing of the conduits 1429, similar to the gas distribution assembly 330. By way of example, the gas distribution assembly may include a support, such as a spring, a woven metal or plastic hose, or a filler (e.g., the filler 350), coupled to and extending along the length of the conduit 1429. The support is configured resist collapsing of the conduit 1429 (e.g., by applying a biasing force outward from the gas distribution volume 1422, etc.). By way of another example, the tubes 1424 may be made from a rigid material. In such an embodiment, the connections between the tubes 1424, the tee fittings 1426, and the elbow fittings 1428 may be free to swivel or rotate, making the gas distribution assembly 1420 more flexible. In other embodiments, the entire gas distribution assembly 1420 is rigid. By way of yet another example, the conduit 1429 may be configured to inflate when supplied with neutralizer by the emitter. The tubes 1424 may be airtight, and the outlets 1430 may be spaced and sized such that the pressure of the neutralizer within the conduit 1429 imparts a biasing force that resists collapsing of the conduit 1429.

The conduits 1429 may be removably or fixedly coupled to the panels (e.g., the top panel 1402, the left panel 1406, the right panel 1408, the front panel 1410, and the rear panel 1412). By way of example, the conduits 1429 may be hung on hooks, attached with a fastener that loops around the conduit 1429 (e.g., cable ties, string, etc.), attached with a hook and loop fastener, or using another type of fastener. As shown in FIGS. 28 and 29, the conduits 1429 are contained within the enclosure 1400. In other embodiments, the conduits 1429 may be contained within the enclosure 1400, may be positioned entirely outside of the enclosure 1400, or a portion of a conduit 1429 may extend into the enclosure 1400. By way of example, the conduit 1429 may be positioned outside of the enclosure 1400, and the tee fittings 1426 may extend through the panels of the enclosure 1400 such that the outlets 1430 are in fluid communication with the enclosed volume 1414

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the terms “exemplary” and “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent, etc.) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the systems as shown in the exemplary embodiments is illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claim.

What is claimed is:

1. A bag, comprising:
at least one panel defining:
an enclosed volume configured to receive at least one item; and
an opening configured to at least selectively facilitate access to the enclosed volume;
an emitter configured to provide a supply of neutralizer; and
a gas distribution assembly coupled to the at least one panel, the gas distribution assembly defining a gas distribution volume extending along the at least one panel and a plurality of apertures positioned along a length of the gas distribution volume and configured to fluidly couple the gas distribution volume to the enclosed volume;
wherein the gas distribution volume is fluidly coupled to the emitter such that the neutralizer is configured to flow from the emitter, through the gas distribution volume, and into the enclosed volume.

2. The bag of claim 1, wherein the at least one panel further defines a top side, a bottom side, and a third side of the bag, wherein the third side extends between the top side and the bottom side, wherein the opening is accessible when the bottom side of the bag is placed on a support surface, and wherein the gas distribution volume extends along at least one of the top side and the third side.

3. The bag of claim 2, wherein the gas distribution volume extends along both the top side and the third side.

4. The bag of claim 1, wherein the gas distribution assembly and the at least one panel coupled to the gas distribution assembly are flexible such that the bag is configured to be at least partially collapsed.

5. The bag of claim 4, wherein the gas distribution assembly further includes a conduit that defines the gas distribution volume, wherein the apertures are positioned along a length of the conduit, and wherein the gas distribution assembly is configured to resist collapsing of the conduit.

6. The bag of claim 5, wherein the gas distribution assembly includes a support extending along the length of the conduit, wherein the support is configured to resist collapsing of the conduit.

7. The bag of claim 1, wherein the emitter includes an ozone generator configured to provide ozone, and wherein the neutralizer includes the ozone.

8. The bag of claim 1, wherein the gas distribution volume forms a continuous loop.

9. The bag of claim 1, further comprising a pocket coupled to the at least one panel, wherein the enclosed volume includes a main volume in direct fluid communication with the gas distribution assembly and a secondary volume defined by the pocket, wherein the at least one panel defines a second opening configured to facilitate access to the secondary volume, and wherein the secondary volume is fluidly coupled to the main volume such that the neutralizer is configured to pass from the main volume into the secondary volume.

10. The bag of claim 1, wherein the at least one panel includes a pair of panels coupled to one another by a seam, wherein the gas distribution assembly includes a duct that at least partially defines the gas distribution volume, wherein the apertures are positioned along the length of the duct, and wherein the duct extends into the seam.

11. The bag of claim 1, further comprising a filter coupled to the at least one panel and extending within the enclosed volume, wherein the filter is configured to passively adsorb odor particles from the enclosed volume.

12. An assembly for neutralizing substances or organisms, comprising:
an enclosure defining an enclosed volume, the enclosure having a top side, a bottom side, and a third side extending between the top side and the bottom side;
an emitter configured to provide a supply of neutralizer; and
a gas distribution assembly including a conduit extending within the enclosure and coupled to the enclosure, the conduit defining a gas distribution volume extending along at least one of the top side and the third side of the enclosure, wherein the gas distribution assembly further defines a plurality of apertures positioned along a length of the conduit that fluidly couple the gas distribution volume to the enclosed volume;
wherein the gas distribution volume is fluidly coupled to the emitter such that the neutralizer is configured to flow from the emitter, through the gas distribution volume, and into the enclosed volume.

13. The enclosure of claim 12, wherein at least a portion of the conduit is flexible, wherein the gas distribution assembly further includes a support extending along the length of the conduit and configured to resist collapsing of the conduit.

14. The enclosure of claim 13, wherein the gas distribution volume forms a continuous loop, and wherein the gas distribution volume extends along both the top side and the third side.

15. The enclosure of claim 14, wherein the emitter includes an ozone generator configured to provide ozone, and wherein the neutralizer includes the ozone.

16. The enclosure of claim 15, further comprising a filter coupled to the enclosure and in fluid communication with the enclosed volume, wherein the filter is configured to passively adsorb odor particles from the enclosed volume.

17. A neutralizing system, comprising:
an emitter including an ozone generator configured to provide a supply of ozone; and
a gas distribution assembly including a conduit, the conduit defining a gas distribution volume extending along a length of the conduit, wherein the gas distribution assembly defines a plurality of outlets fluidly coupled to the gas distribution volume and positioned along the length of the conduit;
wherein the gas distribution volume is fluidly coupled to the emitter such that the gas distribution assembly is configured to expel the ozone from the gas distribution volume at multiple locations along the length of the conduit.

18. The neutralizing system of claim 17, wherein at least a portion of the conduit is flexible, wherein the gas distribution assembly includes a filler positioned within the gas distribution assembly, and wherein the filler is configured to apply a biasing force on the conduit outward from the gas distribution volume to resist collapsing of the conduit.

19. The neutralizing system of claim 18, wherein the conduit includes a sheet of material folded over and engaging itself to form the gas distribution volume, wherein the sheet of material forms a flange portion that is configured to be inserted into a seam.

20. The neutralizing system of claim 17, wherein the gas distribution assembly is coupled to at least one of a bed, a pad, a tent, a seat cover, and a wearable device.
